(19) United States
(12) Patent Application Publication
(10) Pub. No.: US 2012/0219576 A1
(43) Pub. Date: Aug. 30, 2012

(54) LASSA VIRUS-LIKE PARTICLES AND METHODS OF PRODUCTION THEREOF

(75) Inventors: Luis M. Branco, New Orleans, LA (US); Robert F. Garry, New Orleans, LA (US)

(73) Assignee: THE ADMINISTRATORS OF THE TULANE EDUCATIONAL FUND, New Orleans, LA (US)

(21) Appl. No.: 13/395,777

(22) PCT Filed: Sep. 15, 2010
(86) PCT No.: PCT/US10/48972

§ 371 Date: May 16, 2012

Related U.S. Application Data
(60) Provisional application No. 61/243,016, filed on Sep. 16, 2009.

(51) Int. Cl. A61K 39/12; A61P 37/04; C12Q 1/70; C12N 15/63; C12P 21/00

(52) U.S. Cl. 424/186.1; 435/320.1; 435/69.1; 435/5

(57) ABSTRACT

The instant invention is directed to novel Lassa virus-like particle (VLP) compositions and methods of production thereof. The inventive VLPs comprise, for example, the Lassa virus (LASV) Z matrix protein, glycoproteins (GPs)-I and -2, and nucleoprotein (NP). A novel method for producing these VLPs comprises constructing multicistronic plasmids for the expression of VLP protein components from a single vector. One example is a tricistronic vector containing DNA sequences encoding the LASV Z, GPC and NP proteins. The VLPs provided by the present invention can be used for research, therapeutic and diagnostic purposes.

This application claims the benefit of priority to U.S. Provisional Application No. 61/243,016, filed Sep. 16, 2009, which is herein incorporated by reference in its entirety.

### STATEMENT REGARDING FEDERALLY SPONSORED RESEARCH OR DEVELOPMENT

This invention was made, in part, with support provided by the U.S. government under Grant Nos. 1 UC1 AI067188-01 and 1U01AI082119-01 awarded by the National Institute of Allergy and Infectious Diseases of the National Institutes of Health. The Government has certain rights in this invention.

### FIELD OF THE INVENTION

The instant invention relates to the preparation of arenavirus-like particles (AVLPs), particularly Lassa virus-like particles (VLPs), for providing a safe and effective source of viral antigens. The invention can be used for research and medical purposes.

### BACKGROUND OF THE INVENTION

Lassa is an often-fatal hemorrhagic illness named for the town in the Yedseram River valley of Nigeria in which the first described cases occurred in 1969. Parts of Sierra Leone, Guinea, Nigeria, and Liberia are endemic for the etiologic agent, Lassa virus (LASV). The public health impact of LASV in endemic areas is immense. It has been estimated that there are up to 300,000 cases of Lassa per year in West Africa and 5,000 deaths. In some parts of Sierra Leone, 10-15% of all patients admitted to hospitals have Lassa. Case fatality rates for Lassa are typically 15% to 20%, although in epidemics overall mortality can be as high as 45%. The mortality rate for women in the last month of pregnancy is always high, ˜90%, and LASV infection causes high rates of fetal death at all stages of gestation. Mortality rates for Lassa appear to be higher in non-Africans, which is of concern because Lassa is the most commonly exported hemorrhagic fever. Because of the high case fatality rate, the ability to spread easily by human-human contact and potential for aerosol release, LASV is classified as a Biosafety Level 4 and NIAID Biodefense category A agent.

Lassa Virus

LASV is a member of the Arenaviridae family. The genome of arenaviruses consists of two segments (Large, L and Small, S) of single-stranded, ambisense RNA. The enveloped virions (diameter: 110-130 nm) contain two glycoproteins GP1 and GP2 (expressed from a precursor referred to as GPC) and a single nucleoprotein NP (FIG. 1). Electron micrographs of arenaviruses show grainy particles that are ribosomes acquired from the host cells. Hence, use of the Latin “arena,” which means “sandy” for the family name. The arenaviruses are divided into two groups, the Old World or lymphocytic choriomeningitis virus (LCMV)/LASV complex and the New World or Tacaribe complex. Other arenaviruses that cause illness in humans include Junin virus (Argentine hemorrhagic fever, AHF), Machupo virus (Bolivian HF), Guanarito virus (Venezuelan HF) and Sabiá virus (Brazilian HF). Arenaviruses are zoonotic; each virus is associated with a specific species of rodent. The reservoir of LASV is the “multimammate rat” of the genus Mastomys. Mastomys species show no symptoms of LASV infection, but shed the virus in saliva, urine and feces. The wide distribution of Mastomys in Africa makes eradication of this rodent reservoir impractical and ecologically undesirable. Mastomys species often live in human homes and the virus is easily transmitted to humans. Transmission occurs via direct contact with rat urine, feces, and saliva, or by contact or ingestion of excretion-contaminated materials. Infection may also occur when Mastomys species are caught and prepared as food. LASV is readily transmitted between humans, via exposure to blood or bodily fluids, making nosocomial infection a matter of great concern. The stability of the virus in aerosol, plus the ability of the virus to infect guinea pigs and monkeys via the respiratory route, emphasize the potential for aerosol transmission of LASV in bioterrorism or other settings.

Lassa

Signs and symptoms of Lassa, which occur 1-3 weeks after virus exposure, are highly variable, but can include fever, retrosternal, back or abdominal pain, sore throat, cough, vomiting, diarrhea, conjunctival injection, and facial swelling. LASV infects endothelial cells, resulting in increased capillary permeability, diminished effective circulating volume, shock, and multi-organ system failure. Frank bleeding, usually mucosal (gums, etc.), occurs in less than a third of cases, but confers a poor prognosis. Neurological problems have also been described, including hearing loss, tremors, and encephalitis. Patients who survive begin to defervesce 2-3 weeks after onset of the disease. Temporary or permanent unilateral or bilateral deafness occurs in ˜30% of Lassa fever patients; these effects are not associated with the severity of the acute disease. The antiviral drug ribavirin is effective in the treatment of Lassa fever, particularly early in the course of illness. Maintenance of appropriate fluid and electrolyte balance, oxygenation and blood pressure may also improve survival. Passive transfer of neutralizing antibodies early after infection may also be an effective treatment for Lassa and other arenaviral hemorrhagic fevers. No LASV vaccine is currently available.

Lassa Vaccine Development Efforts

Development of an effective LASV vaccine is crucial both for bioterrorism preparedness and to improve public health in endemic areas. In prior vaccine studies (Table 1) the two major animal models used for LASV challenge have been non-human primates (NHP) and guinea pigs.

TABLE 1
Prior Lassa virus vaccine studies

| | Animal | Routes | | |
|---|---|---|---|---|
| | challenge | vaccine/ | | |
| Vaccine platform | model | challenge | Results | Reference |
| gamma radiation- | Rhesus | IM 1X | No protection | McCormick et al., |
| inactivated LASV | macaques | | | 1992 |
| gamma radiation- | Papio | IM 1X | Complete protection IM; | Krasnianski [symbol] et al., |
| inactivated LASV | hamadryas | | 50% respiratory | 1993 |
| LASV/rVV¹ | Rhesus and | ID 1X/IM | 88 | Fisher-Hoch et al., |
| GP1and 2 GP1, 2, | Cynomolgus | | 3/15 (20% survival) | 1989 |
| and NP | macaque | | | |
| LASV NP/rVV | | | | |
| LASV | Cynomolgus | IM1x/IM | Complete protection | Giesbert et al., 2005 |
| GPC/rVSV² | macaque | | | |
| LASV NP/rVV | Guinea pig | ID 1X | Complete protection | Clegg and Lloyd, |
| | | | | 1987³ |
| LASV GPC or | Guinea pig | | Complete protection with | Pushco et al., 2001 |
| NP/VRP⁴ | | | either GP or NP | |
| LASV GPC/ | Guinea pig | | Complete protection | Bredenbeek |
| YFV17D⁵ | | | | et al., 2006 |
| Mopeia-LASV | Guinea pig | | Complete protection | Carrion et al., 2007 |
| live attenuated | | | | |

¹recombinant vaccina virus;
²recombinant vesicular stomatitis virus;
³similar results reported by Morrison et al., 1989;
⁴Venezualian equine encephalitis virus-like replicon particles;
⁵yellow fever virus vaccine strain 17D

Both develop fatal infections with LASV. As expected, the disease in NHP more closely resembles that of humans. In a limited study (three immunized NHP given a single dose), gamma-radiation-inactivated LASV failed to protect rhesus macaques. However, another study found that inactivated LASV protected another species of NHP after a single injection. Recombinant vaccinia virus (rVV) expressing LASV glycoproteins protected both guinea pigs and macaques; both GP1 and GP2 were necessary to confer protection. A rVV vector expressing only NP was protective in guinea pigs, but not NHP. In these prior studies, it has been observed generally that protection from LASV challenge did not correlate with the magnitude of the humoral immune response. For example, antibodies against LASV structural proteins were induced in a study in which gamma-radiation-inactivated LASV failed to protect from lethal challenge. Collectively, these prior studies indicate that induction of cellular immune responses may be critical for protection from fatal Lassa disease. Innate immune responses may also be involved. An attenuated reassortant virus, which has the L genome segment from Mopeia virus (a non-lethal arenavirus) and the S genome segment from LASV, and thus expresses LASV glycoproteins, protected both mice and guinea pigs from Lassa fever challenge. Remarkably, this vaccine delivered on the same day as the LASV challenge protected 7 of 9 guinea pigs. The effectiveness of passive immunotherapy with Lassa fever immune plasma (LFIP) in suspected cases of febrile hemorrhagic fever has not been established, and many accounts are anecdotal and poorly characterized. However, similar studies performed in NHP with LFIP have proven highly effective in protecting against lethal challenge with LASV, especially if the treatment is administered early in infection.

Live viral vaccines have traditionally offered the most effective long-term protection against LASV, in part because they deliver antigen endogenously and are effective at inducing antigen-specific activation of CD8 T-cells. Subunit vaccines typically have not provided as much durability, presumably because exogenous antigens are typically taken up by antigen-presenting cells (APCs) via phagocytic or endocytic processes and activate antigen-specific CD4 T-cells. Fortunately, modern adjuvants (e.g., ADP-ribosylating protein adjuvants and Toll-like receptor [TLR] agonists) and new delivery strategies (e.g., mucosal and transcutaneous immunization) can be effective at activating both CD4 and CD8 T-cells to exogenous antigens. These adjuvants and delivery systems have been shown to induce both humoral and cellular responses against a number of exogenous proteins, broadening the immune repertoire to include both neutralizing antibodies and cytotoxic T lymphocyte (CTL) responses. While neutralizing antibodies have not historically been a dominant protective factor in preventing LASV infections, it has been suggested that they function synergistically with CTL responses against LASV and other arenaviruses. Moreover, LASV neutralizing antibodies do not typically develop until late in convalescence and it is unknown if pre-existing high-titer virus-neutralizing antibodies induced by vaccination would have a major impact on infectivity.

The VLP (virus-like particle) platform is quickly emerging as a highly viable alternative for the generation of viral vaccines, with improved safety and immunogenicity profiles. The worldwide launch of Gardasil®, a tetravalent, VLP-based human papillomavirus (HPV) vaccine produced in yeast, by Merck & Co. in 2007, has been remarkably safe and well tolerated, with very few reported serious side effects in millions of vaccinations to date. Novavax has recently completed enrollment of healthy volunteers in a Phase IIa clinical trial of its VLP-based seasonal influenza vaccine. The vaccine is produced in a baculovirus expression system, and yields are reportedly significantly higher than in egg- or mammalian cell-based platforms. LigoCyte's lead vaccine candidate is being developed for the prevention of norovirus infection in humans, a temporarily debilitating illness that afflicts millions of individuals annually. In addition, LigoCyte is using a similar VLP platform for the development of its seasonal influenza vaccine. Recently, VLP-based vaccines against Ebola and Marburg viruses have been tested in NHP and found to be fully protective.

Given practical limitations of live vaccines, there is interest in developing replication incompetent VLP-based vaccines for LASV. However, it is not known if immunity to replication incompetent LASV VLPs would preferentially induce a humoral or cellular response, or both. Future VLP-based vaccine formulations and administration regimens for treating Lassa will depend on the preferential immune response (humoral versus cellular), and will be aimed at generating a robust and long-term protection profile against LASV. Since non-live vaccines are easier to produce, store, and deliver than live vaccines, and may be safer in areas where LASV is endemic, this will be an important developmental step for producing effective vaccines against LASV as well as other biothreat agents. In response to this need, the present invention provides novel VLP compositions and methodology for enhanced VLP production.

### SUMMARY OF THE INVENTION

One embodiment of the invention is drawn to a nucleic acid expression construct for producing an arenavirus-like particle. This construct can comprise sequences encoding (i) a first protein comprising an arenavirus matrix (Z) protein or functional fragment or variant thereof, and (ii) at least a second protein comprising or consisting of a different arenavirus protein or functional fragment or variant thereof. The arenavirus protein-encoding sequences of the construct are capable of being expressed in a eukaryotic cell. The second protein of the construct can comprise or consist of an arenavirus glycoprotein precursor (GPC) protein or functional fragment or variant thereof, an arenavirus nucleoprotein (NP) or functional fragment or variant thereof, an arenavirus glycoprotein-1 (GP1) protein or functional fragment or variant thereof, or an arenavirus glycoprotein-2 (GP2) protein or functional fragment or variant thereof. Another embodiment of the nucleic acid expression construct can comprise a sequence encoding a third protein, wherein the second and third proteins encoded by the sequences comprise, respectively, arenavirus GPC and NP proteins, or functional fragments or variants thereof In another embodiment, the first protein comprises or consists of an arenavirus Z protein and the second protein comprises or consists of an arenavirus NP, GPC, GP1, or GP2 protein.

The inventive nucleic acid construct may comprise at least one sequence that encodes a protein derived from LASV. Alternatively, all of the arenavirus proteins encoded by the sequences of the construct are derived from LASV. In one embodiment, the Z, NP, GPC, GP1 and/or GP2 proteins encoded by the construct are derived from LASV.

With the inventive nucleic acid construct, each arenavirus protein-encoding sequence may be comprised within its own expression cassette. Each expression cassette may contain a promoter and/or a transcription termination sequence. In a preferred embodiment, the expression construct is a eukaryotic expression construct/vector.

Another embodiment of the instant invention is directed to a method of preparing an arenavirus-like particle having the steps of providing a nucleic acid expression construct as discussed above, and introducing this construct into a eukaryotic cell to express the first and second proteins encoded by the sequences of the construct. With this method, the expressed first and second proteins may organize into arenavirus-like particles that bud from the membrane surface of the cell into which the construct was introduced. A preferred embodiment of this method employs a mammalian cell. In another embodiment, all of the arenavirus proteins encoded by the construct are derived from LASV. This method employs expression of LASV proteins in cis (i.e., in the same cell).

The instant invention is also drawn to an arenavirus-like particle having (i) a first protein comprising or consisting of an arenavirus matrix (Z) protein or functional fragment or variant thereof, and (ii) a second protein comprising or consisting of an arenavirus nucleoprotein (NP) or functional fragment or variant thereof. The arenavirus-like particle may also comprise a third protein comprising or consisting of an arenavirus glycoprotein precursor (GPC) protein or functional fragment or variant thereof; an arenavirus glycoprotein-1 (GP1) protein or functional fragment or variant thereof, or an arenavirus glycoprotein-2 (GP2) protein or functional fragment or variant thereof In another embodiment, the first protein comprises or consists of an arenavirus Z protein, the second protein comprises or consists of an arenavirus NP, and the third protein comprises or consists of GPC, GP1, or GP2. These VLP particles of the invention may be produced according to the above method.

The inventive arenavirus-like particle may comprise at least one protein derived from LASV. Alternatively, all of the arenavirus proteins of the particle are derived from LASV. In one embodiment, the Z, NP, GPC, GP1 and/or GP2 proteins of the particle are derived from LASV. Vaccines comprising the inventive arenavirus-like particles are also part of the instant invention; preferred embodiments thereof comprise Lassa VLPs.

### BRIEF DESCRIPTION OF THE FIGURES

FIG. 1: Structure of the Lassa virus virion.

FIG. 2: Amino acid sequence (SEQ ID NO:1) and corresponding DNA coding sequence (SEQ ID NO:2) of LASV Z matrix protein.

FIG. 3: Map of pcDNA3.1+zeo:intA showing certain restriction endonuclease sites.

FIG. 4: Amino acid sequence (SEQ ID NO:9) and corresponding DNA coding sequence (SEQ ID NO:10) of LASV NP protein.

FIG. 5: Amino acid sequence (SEQ ID NO:12) and corresponding DNA coding sequence (SEQ ID NO:13) of LASV GPC protein.

FIG. 6: Tricistronic pcDNA3.1+zeo:intA:LASV GPC+NP+Z construct for the expression and assembly of LASV VLP in mammalian cells.

FIG. 7: Complete DNA sequence (SEQ ID NO:8) generated in silico of an example of a tricistronic construct using pcDNA3.1+zeo:intA vector as the backbone for expression of LASV GPC+NP+Z to produce VLPs. Refer to Example 2. intA sequences are bounded by GT (5′ side) and AG (3′ side) prototypical intron border dinucleotides as shown with double-underlining. Certain restriction endonuclease sites and primer sites discussed in the application are identified with underlined, bold and/or italicized characters.

FIG. 8A and B: Analysis of HEK-293T cell-generated Lassa VLPs: purification by PEG-6000 precipitation, followed by sucrose gradient centrifugation, and detection of Z- and GP (GPC)-containing fractions by SDS-PAGE and western blot analyses. (A) top panel, western blots probed for LASV GP1 (top panel) and His-tag (bottom panel, LASV Z protein). (B) diagrammatic representation of the sucrose gradient as observed post centrifugation. Red bands depict certain protein pellets.

FIG. 9: Analysis of HEK-293T cell-generated Lassa VLPs in small scale transfections: For each VLP western (second and fourth blots from the top), 1-10 μg VLPs were loaded. VLPs were obtained by the below-described sucrose centrifugation protocol (briefly, PEG-6000 precipitation followed by sucrose gradient centrifugation.

FIG. 10: Mouse IgG (γ) endpoint titer to Lassa VLP (Z+GPC). ELISA analysis was used to measure the serum level of anti-LASV IgG antibodies in mice immunized with Lassa VLP (Z+GPC). Lassa VLP (Z+GPC) was the target antigen coated on the ELISA plates. Refer to Example 4 for additional details. rLASV, recombinant LASV.

FIG. 11: Mouse IgG+IgM+IgA endpoint titer to Lassa VLP (Z+GPC). ELISA analysis was used to measure the serum level of anti-LASV IgG, IgM and IgA antibodies in mice immunized with Lassa VLP (Z+GPC). Lassa VLP (Z+GPC) was the target antigen coated on the ELISA plates. Refer to Example 4 for additional details. rLASV, recombinant LASV.

FIG. 12: Mouse IgG (γ) endpoint titer to Lassa GP1 and GP2. ELISA analysis was used to measure the serum level of anti-LASV IgG antibodies in mice immunized with Lassa VLP (Z+GPC). Lassa sGP1 and sGP2 were the target antigens coated separately on the ELISA plates. Refer to Example 4 for additional details. rLASV, recombinant LASV; sGP1, soluble GP1; sGP2, soluble GP2.

FIG. 13: Purification of HEK-293T/17-generated LASV VLP by sucrose gradient sedimentation, and detection of GP1, GP2, NP, and Z proteins in fractions by western blot analysis (Example 5). LASV VLP were precipitated with PEG-6000/NaCl and concentrated by ultracentrifugation. Pellets were resuspended in 500 μL of THE or PBS, overlaid on discontinuous 20-60% sucrose gradients, and sedimented by ultracentrifugation. Eight fractions of 500 μL each were collected from sucrose gradients. Ten μL from each fraction was separated on denaturing 10% NuPAGE® gels, blotted and probed with LASV protein-specific mAbs. LASV VLP containing Z+GPC+NP (A) and Z+GPC (B) were analyzed for distribution of GP1 (Ai, Bi), GP2 (Aii), NP (Aiii), and Z (Aiv, Bii) throughout the gradient spectrum. Fraction 1 contained input supernatant (S) loaded onto gradients (lane 1). Fractions 2 through 8 were from 20-60% sucrose gradients. Lane 9 contained insoluble material that pelleted through 60% sucrose (P). The size of each protein in kDa is indicated to the right of each blot (unprocessed GPC: 75 kDa, GP1: 42 kDa, GP2: 38 kDa, NP: 60 kDa, and Z: 12 kDa).

FIG. 14: Light microscopy analysis of HEK-293T/17 cells transfected with LASV gene constructs (Example 5). Representative fields of untransfected or vector control-transfected (A), LASV NP or GPC (B), or Z, Z+GPC, Z+NP, Z+GPC+NP (C) transfected HEK-293T/17 cells at 72 hours photographed in 6-well plates at 400× magnification are shown.

FIG. 15: Lectin binding profiles on sucrose purified VLP (Example 5). LASV Z+GPC+NP VLP fractions obtained from sucrose gradient sedimentation corresponding to those in FIG. 13A were subjected to SDS-PAGE and lectin binding analysis on proteins transferred to nitrocellulose membranes (A). A combination of agglutinins, GNA (Galanthus nivalis), SNA (Sambucus nigra), MAA (Maackia amurensis), PNA (Peanut), and DSA (Datura stramonium), were combined and used to probe VLP fractions 1 through 9 (A, lanes 1-9). LASV NP, GP1, and GP2 generated in E. coli were used as unglycosylated protein controls (A, lane 10). A combination of four glycoproteins was used as positive controls for lectin binding: carboxypeptidase Y (63 kDa), transferrin (80 kDa), fetuin (68, 65, 61 kDa), and asialofetuin (61, 55, 48 kDa) (A, lane 11). For visual comparison purposes, an SDS-PAGE gel was run with the same VLP fractions, stained with Coomassie BB-R250, and photographed (B, lanes 1-9). LASV Z, Z+GPC+NP, Z+GPC, and Z+NP VLP purified through 20% sucrose cushions were similarly analyzed for glycan binding (C, lanes 1-4, respectively). The relative positions of GPC, GP1, and GP2 are noted to the left of the gel. Protein molecular weights in kDa are noted to the right of each image.

FIG. 16: Deglycosylation analysis of LASV Z+GPC+NP VLP (Example 5). Non-denatured LASV Z+GPC+NP VLP were subjected to deglycosylation with PNGase F (A-D, lane 2), Endo H (A-D, lane 3), Neuraminidase (A-D, lane 4), or were left untreated (A-D, lane 1), followed by SDS-PAGE and western blot analyses. Blots were probed with α-GP1 (A), α-GP2 (B), α-6X-HIS (D) mAbs, or α-NP pAb (C). Protein molecular weights in kDa are noted to the right of each blot.

FIG. 17: Analysis of RNA content in LASV VLP and corresponding transfected HEK-293T/17 cells (Example 5). A. RNA was isolated from the total VLP fraction generated in a single 10-cm cell culture dish (˜6×10⁷ cells), and the entire nucleic acid pellet was resolved on denaturing glyoxal agarose gels. RNA from Z3′HIS, Z3′HIS+GPC, Z3′HIS+NP, Z3′HIS+GPC+NP, and Z+GPC+NP VLP (lanes 2, 4, 6, 8, and 10, respectively [V]), and 5 μg of total RNA isolated from the corresponding transfected HEK-293T/17 cells (lanes 1, 3, 5, 7, and 9, respectively [C]) were resolved per lane of a 1.5% gel. Untransfected HEK-293T/17 cell RNA was run alongside test samples as a control (lane 11 [C]). Molecular weight sizes ranging from 0.5-6 kbp are noted to the left of the gel. The positions of cellular 28S and 18S ribosomal RNAs, and tRNA are noted to the right of the gel. B. A separate western blot analysis revealed that input LASV VLP expressed the respective proteins of interest (lanes 2, 4, 6, 8, 10).

FIG. 18: Electron micrographs of LASV VLP budding from the surface of HEK-293T/17 cells expressing LASV Z alone or in combination with GPC and NP genes (Example 5). Cells expressing LASV Z (A), Z+NP (B), or Z+NP+GPC (C) were harvested at 72 hours post transfection, fixed in glutaraldehyde, and embedded in agarose plugs. Cell pellets were processed for EM analysis and were imaged. LASV VLP budding from the surface of transfected cells or approaching the cell surface are marked by black arrows. The bar in each figure equals 100 nm.

FIG. 19: Trypsin protection assay on LASV Z+GPC+NP VLP (Example 5). LASV VLP expressing Z, GPC, and NP proteins were subjected to trypsin protection assays to assess the enveloped nature of pseudoparticles and compartmentalization of viral proteins. LASV VLP incorporated unprocessed 75 kDa GPC precursor (A-B, lane 1), and monomeric 42 kDa GP1 (A, lane 1), and 38 kDa GP2 (B, lane 1). LASV VLP also incorporated trimerized, non-reducible 126 kDa GP1 isoforms (A, lane 1), and 114 kDa GP2 trimers to a lesser extent (B, lane 1). For trypsin protection assays, ten μg of LASV VLP were either left untreated (lane 1), treated with 3 mg/mL soybean trypsin inhibitor (lane 2), 1% Triton® X-100 (lane 3), 100 μg/mL trypsin (lane 4), 1% Triton® X-100 and 100 μg/mL trypsin (lane 5), or 100 μg/mL trypsin in the presence of 3 mg/mL soybean trypsin inhibitor (lane 6). Trypsin treatment of intact VLP did not significantly affect the levels of NP (C, lane 4), and Z (D, lane 4) proteins. Treatment of VLP with Triton® X-100 in the presence of trypsin resulted in the complete digestion of NP (C, lane 5) and Z (D, lane 5), while only partially degrading monomeric GP 1 (A, lane 5) and GP2 (B, lane 5) proteins. Treatment of VLP with trypsin in the presence of soybean trypsin inhibitor completely prevented digestion of any form of all viral proteins (A-D, lane 6).

FIG. 20: Immunogenicity of LASV Z+GPC and Z+GPC+NP in a prime+2 boosts regimen in BALB/c mice (Example 5). Groups of 10 BALB/c mice were immunized i.p. with either 100 μL of sterile TNE, or 10 μg of LASV VLP formulated in the same buffer using a prime+2 boosts regimen, 3 weeks apart. Three weeks after the second boost all mice were sacrificed and sera were subjected to murine IgG endpoint titer determinations by ELISA on homologous VLP or recombinant LASV proteins coated on Nunc Maxisorp® plates. Endpoint titers were calculated using background subtraction binding values generated with normal mouse sera on recombinant VLP and LASV proteins. LASV Z+GPC immunizations generated significant titers against whole VLP (mean=8,445), but generally low titers to viral GP1 and GP2, with means of 238 and 318, respectively (A). A similar immunization schedule with LASV Z+GPC+NP VLP resulted in significantly higher endpoint titers to both glycoproteins, with mean of 4850 for both (B), and to whole VLP (mean=25,600). Significant IgG titers were also generated to NP (mean=1,600). Endpoint titers generated by sham immunized murine sera to recombinant LASV proteins were at the lower limit of detection of the assay (mean=10), with slight increased non-specific titers against Z+GPC VLP (mean=18) and Z+GPC+NP (mean=50). The immunization schedule used in these experiments is graphically outlined in C.

FIG. 21: Binding profile of human serum IgM and IgG, and NP-specific mAbs on LASV VLP and recombinant nucleoprotein (Example 5). Human sera collected from household contacts of patient G676, individuals hospitalized at the KGH at the time of analysis, or from supposedly LASV naive controls were diluted 1:100 in a proprietary sample diluent buffer containing 0.05% Tween® 20 (Corgenix Medical Corp.) and assayed by ELISA on plates coated with 2 μg/mL total VLP protein (A, C) or 2 μg/mL rNP (B, D) per well. Detection of bound human IgM (A, B) or IgG (C, D) was performed as outlined in methods. LASV VLP captured IgM from three samples (G676-M, G676-Q, G688-1), all of which were also detected by rNP ELISA (A, B), but did not result in binding by IgM from 14 additional samples that also tested positive on rNP (A, B), including the G652-3 positive control. Similarly, VLP detected LASV-specific IgG in 2 samples (G679-2, G679-3), but did not identify 24 others detected in rNP ELISA (C, D). For analysis of mAb binding profiles, LASV VLP were coated in high protein binding ELISA plates at the same concentration as above. The indicated NP-specific mAbs were then used in a binding assay at 1 μg/mL alongside mouse IgG as a negative control (E). For capture and detection of NP in solution, each NP-specific mAb was coated on ELISA plates at 5 μg/mL, followed by incubation with serial dilutions of nucleoprotein in sample diluent (F). Captured NP was detected with a polyclonal goat α-NP-HRP conjugate.

FIG. 22: Western blot analysis of LASV antigen positive and negative patient sera and controls for GP1, GP2, and NP proteins (Example 6). Twenty μL of a 1:4 dilution of precipitated suspected LASV patient sera was resolved per lane of a reducing SDS-PAGE gel, blotted and probed with α-GP1, α-GP2, or α-NP antibodies. In three samples NP, GP1, GP2 were detected, indicating presence of LASV virions (G692-1, G762-1, G765-1; NP, GP1,2 (+)). In G610-3, G676-A, G583-1, and G755-1, only GP1 was detected (GP1 (+)). Only low levels of NP were detected in G787-1 (NP (+)). Whereas in G337-1 and G079-3, only GP1 was detectable (GP1 (+)); GP2 levels were not determined (nd). Similarly, in G090-3, only low levels of NP were detected, but GP2 levels were not determined (nd). A representative suspect LASV patient serum sample that did not reveal detectable levels of all three viral antigens (G543-3) is shown alongside normal uninfected controls (BOM011, BOM019) (NP, GP1,2 (−)). In vitro controls derived from transfection of HEK-293T/17 cells with pcDNA vector (pcDNA) or a LASV GPC construct harvested at 36 hours (GPC 36 h) are shown. Soluble GP1 can be precipitated from the supernatants of cells expressing GPC (GPC 36 h). LASV VLP containing NP, GP1, and GP2 are shown for protein size comparison (L VLP) (in vitro ctrls). Molecular weights for each LASV protein are shown to the left of blots, and identified on the right.

### DETAILED DESCRIPTION OF THE INVENTION

An important limitation of most live recombinant virus vaccines is the potential for prior immunity to the vector either through natural exposure or prior immunization. Another critical issue is safety, particularly in patient populations in Africa where AIDS and other immunosuppressive illnesses are common. The general handling (e.g., culture and administration) of live viruses also presents a significant risk. The instant invention overcomes these limitations of live vaccines by providing VLP-based vaccines for LASV. Rationale for pursuing a VLP approach include the following factors:
Recombinant VLP-based vaccines are expected to be safe in military and civilian populations, including those in areas endemic for Lassa.
There have been major advances in adjuvant technology. Modern adjuvants such as LT(R192G) (mutant Escherichia coli labile toxin), monophosphoryl lipid A (MPL), and CpG can be used to enhance T-cell responses.
Respiratory infection is the most likely route of LASV infection with respect to its use as a bioterrorism agent, and may be important both in hospital-acquired (nosocomial) and natural infections. Recombinant VLP-based vaccines can be targeted directly to the mucosal surfaces of the respiratory system.
Recombinant LASV GP1, GP2, GPC and NP produced in bacterial or mammalian cells are potent immunogens (Illick et al., 2008, Virol J. 5:161; Branco et al., 2008, Virol J. 5:74, both of which references are herein incorporated by reference in their entirety). The advanced mammalian expression systems produce LASV proteins likely folded in native configurations.
Recombinant antigen-based diagnostic assays for LASV have been developed by the inventors, which will be useful for evaluating LASV vaccine efficacy.
The inventors have established a clinical research program in Sierra Leone, which is an area endemic for LASV. This clinic constitutes a unique resource for future human clinical trials testing Lassa VLP-based vaccines.

VLPs, methods of preparing VLPs, immunogenic compositions that include VLPs, and methods of eliciting an immune response using immunogenic compositions that include VLPs are herein disclosed. The instant invention as described herein is applicable to all arenaviruses (not just LASV), including Ippy virus, Lujo virus, Lymphocytic choriomeningitis virus (LCMV), Mobala virus, Mopeia virus, Amapari virus, Chapare virus, Flexal virus, Guanarito virus, Junin virus, Latino virus, Machupo virus, Oliveros virus, Paraná virus, Pichinde virus, Pirital virus, Sabiá virus, Tacaribe virus, Tamiami virus and Whitewater Arroyo virus, for example. Preferred embodiments of the invention are drawn to VLPs modeling arenaviruses that are infectious to a human population. All these viruses share a related set of protein components that are employed in the inventive VLPs, namely the Z, GPC and NP proteins. A surprising feature of the instant invention is the inclusion of arenavirus NP in the VLP. Another surprising feature of the invention is the production of VLPs through the provision of a multicistronic DNA expression construct. In general, where the below disclosure refers to LASV in particular, such disclosure equally applies to other arenaviruses, such as those listed above.

General Techniques

The practice of the present invention will employ, unless otherwise indicated, conventional techniques of molecular biology (including recombinant techniques), microbiology, cell biology, biochemistry, and immunology, which are all within the normal skill of the art. Such techniques are fully explained in the literature, such as, for example, Molecular Cloning: A Laboratory Manual, second edition (Sambrook, et al., 1989) Cold Spring Harbor Press; Methods in Molecular Biology, Humana Press; Cell Biology: A Laboratory Notebook (I. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Cabs, eds., 1987); Current Protocols in Molecular Biology (F. M. Ausubel, et al, eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

As used herein, the singular form “a”, “an”, and “the” includes plural references unless indicated otherwise. For example, “a” soluble glycoprotein includes one or more soluble glycoproteins.

Compositions and Methods of the Invention

Generally, the present invention provides novel Lassa VLP compositions/particles, methods for enhanced production thereof; and methods for using these VLPs in diagnosis, detection, and treatment of Lassa. Specifically, this invention provides Lassa VLP compositions/particles comprising Z, GPC and NP protein components of LASV. The invention also provides Lassa VLP compositions/particles comprising Z and NP protein components of LASV. It should be understood that the inventive Lassa VLPs minimally comprise the Z protein. A surprising feature of the invention is the inclusion of NP in the VLP. Each LASV component of the inventive VLPs may comprise additional sequences (i.e., in the form of a fusion protein) such as epitopes for detection purposes. Such additional sequences may be non-arenavirus or non-LASV proteins/peptides. The viral protein components of the inventive VLPs retain characteristics of the native viral proteins allowing for development and production of effective diagnostics, vaccines, therapeutics, and screening tools.

Examples of the LASV Z, GPC and NP proteins that can be incorporated in the instant invention are provided herein (SEQ ID NOs:1, 12 and 9, respectively). Skilled artisans will recognize that other forms of these proteins can likewise be incorporated in the invention, such as proteins in fusion with other non-LASV proteins, proteins varying in sequence due to polymorphisms or synthetic changes, and fragments. Sequences that can be fused to the invention's protein components include, for example, non-LASV signal sequences (a.k.a. signal peptides) for protein transport within cells and/or ultimately secretion from cells (e.g., human IgG signal sequences [heavy or light chains], secreted alkaline phosphatase [SEAP] signal sequence, proopiomelanocortin [POMC] signal sequence). Epitopes that can be fused for ease of detection or purification/isolation purposes can be c-myc, HA, Flag, His, V5, GFP, GST, MBP, LacZ, GUS, S-tag, or Strep-Tag®, all of which are well known in the art. In general, non-LASV sequences for fusion purposes can be derived from any bacterial (e.g., E. coli), or eukaryotic (e.g., mammal, insect, plant, fungus, protist, virus) source. Example embodiments of fusion proteins are those in which an epitope is fused at either the N- or C-terminus of an LASV protein. In other examples, the fusion is only between the C-terminus of an LASV protein (e.g., Z protein) and an epitope (e.g., His tag).

The LASV Z protein (also referred to in the art as LASV matrix protein) employed for the instant invention may comprise or consist of SEQ ID NO:1. Alternatively, the Z protein may comprise or consist of an amino acid sequence that is at least about 70%, 75%, 80%, 85%, 90%, 91%, 92%, 93%, 94%, 95%, 96%, 97%, 98%, or 99% identical to SEQ ID NO:1. Such variants of SEQ ID NO:1 should function or behave (e.g., antibody binding activity, ability to generate VLP without additional arenaviral genes when expressed in mammalian cells) the same as or in a similar manner to SEQ ID NO:1 and/or other known Z proteins. Examples of Z proteins that can be used in the invention are disclosed at the U.S. National Center for Biotechnological Information (NCBI) website (or GenBank) under accession numbers NP_{—}694871, YP_{—}170703, AAT49005, AAV54102, AAT49001, AAT48997, AAC05816 and 073557 (these sequences are herein incorporated by reference in their entirety). Skilled artisans will realize that DNA sequences can be used to express the Z protein component for practicing the invention. For example, a DNA sequence comprising or consisting of SEQ ID NO:2 may be used to express LASV Z protein. Alternatively, a DNA sequence comprising or consisting of a sequence that is at least about 60%, 70%, 75%, 80%, 85%, 90%, 91%, 92%, 93%, 94%, 95%, 96%, 97%, 98%, or 99% identical to SEQ ID NO:2 can be used to express LASV Z protein, just so long that the expressed product functions or behaves (e.g., antibody binding activity, ability to generate VLP without additional arenaviral genes when expressed in mammalian cells) the same as or in a similar manner to the Z protein. Examples of DNA sequences encoding Z proteins that can be used in the invention are disclosed at the U.S. NCBI website (or GenBank) under accession numbers AY179175 (positions 52-351), AY179172 (positions 50-349), AY179171 (positions 48-347) and U73034 (positions 66-365) (these sequences, particularly the regions therein that encode the Z proteins as shown parenthetically following each accession number, are herein incorporated by reference in their entirety).

The LASV Z protein is minimally required to produce VLPs, as disclosed by Strecker et al. (2003, J. Virol 77:10700-10705) which is herein incorporated by reference in its entirety, especially as it relates to protocols for producing Lassa VLPs. As described herein, functional variants and/or fragments of the Z protein may be incorporated in the inventive VLPs, just so long that such variant/fragment maintain the ability to allow VLP production, which includes pinching off of particles (i.e., budding) from the membrane surface of cells prepared to express the inventive VLPs. Although Z protein that has been altered at the C-terminus (e.g., deletion of up to thirty C-terminal amino acid residues, mutation of late domains PTAP and/or PPPY [e.g., residues 81-84 and 94-97, respectively, of SEQ ID NO:1]) reduces VLP production, VLPs are still produced. Thus, such altered forms of Z protein can be utilized in the instant invention. Z protein lacking about the last 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 C-terminal amino acid residues may be employed in the instant invention. These are examples of functional variants/fragments of the Z protein.

The LASV GPC protein employed for the instant invention may comprise or consist of SEQ ID NO:12. Alternatively, the GPC protein may comprise or consist of an amino acid sequence that is at least about 70%, 75%, 80%, 85%, 90%, 91%, 92%, 93%, 94%, 95%, 96%, 97%, 98%, or 99% identical to SEQ ID NO:12. Such variants of SEQ ID NO:12 should function or behave (e.g., antibody binding activity) the same as or in a similar manner to SEQ ID NO:12 and/or other known GPC proteins. Examples of GPC proteins that can be used in the invention are disclosed at the U.S. NCBI website (or GenBank) under accession numbers YP_{—}170705, AAV54104, AAT49014, AAT49012, AAT49010, AAT49008, AAT49004, AAT49000, AAO59512, AAG41802, AAL13212, AAF86703 and AAF86701 (these sequences are herein incorporated by reference in their entirety). Skilled artisans will realize that DNA sequences can be used to express the GPC protein component for practicing the invention. For example, a DNA sequence comprising or consisting of SEQ ID NO:13 may be used to express LASV GPC protein. Alternatively, a DNA sequence comprising or consisting of a sequence that is at least about 60%, 70%, 75%, 80%, 85%, 90%, 91%, 92%, 93%, 94%, 95%, 96%, 97%, 98%, or 99% identical to SEQ ID NO:13 can be used to express LASV GPC protein, just so long that the expressed product functions or behaves (e.g., antibody binding activity, assembling of the glycoprotein tripartite complex comprised of GP1, GP2, and SSP [signal peptide], acquisition of fusogenic properties to target mammalian cells harboring the arenaviral receptor molecule) the same as or in a similar manner to the GPC protein. Examples of DNA sequences encoding GPC proteins that can be used in the invention are disclosed at the U.S. NCBI (or GenBank) website under accession numbers AY179173 (positions 36-1511), AF246121 (positions 54-1529), AF333969 (positions 52-1524), AF181854 (positions 52-1524), AF181853 (positions 52-1524) and X52400 (positions 71-1543) (these sequences, particularly the regions therein that encode the GPC proteins as shown parenthetically following each accession number, are herein incorporated by reference in their entirety).

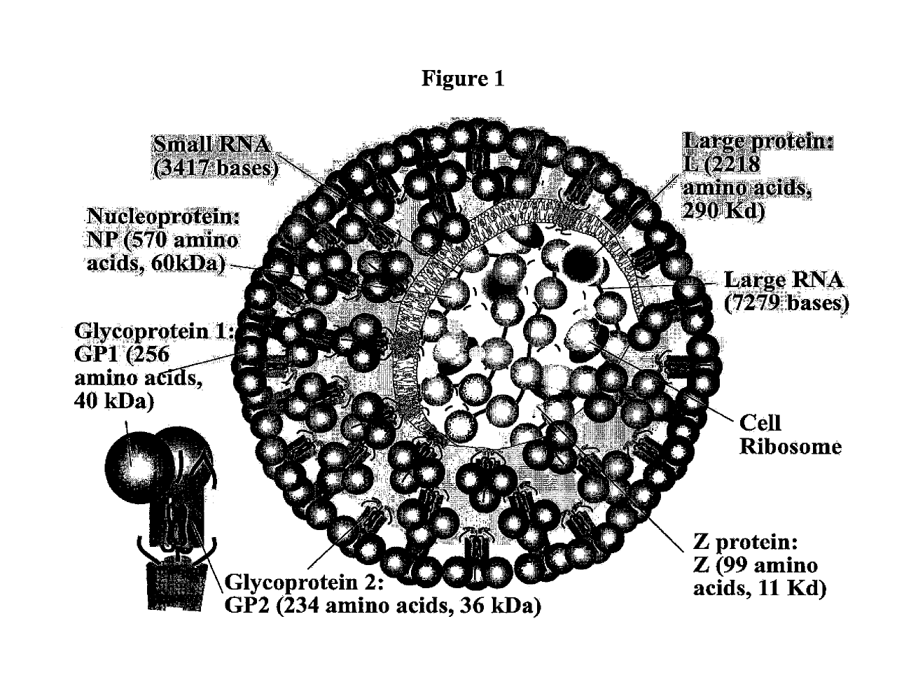

As an alternative to expressing GPC in practicing the instant invention, one can instead express the downstream products thereof (GP1 and GP2) directly. Such could be performed by expressing GP1 (with signal peptide) alone or in combination with GP2. GP2 is preferably co-expressed with GP1 (with signal peptide). Just as with the Z, NP and GPC components, the GP1 and GP2 components could be expressed in the form of fusion proteins (e.g., fusion with a non-LASV sequence) or as functional analogs having at least about 70%, 75%, 80%, 85%, 90%, 91%, 92%, 93%, 94%, 95%, 96%, 97%, 98%, or 99% amino acid residue identity with the GP1 and/or GP2 sequences comprised within SEQ ID NO:12. GP1 is represented by amino acids 1-259 of SEQ ID NO:12 (residues 59-259 represent GP1 cleaved from signal peptide residues 1-58), whereas GP2 is represented by amino acids 260-491 of SEQ ID NO:12. Residues 427-451 and residues 452-491 of SEQ ID NO:12 represent, respectively, the transmembrane domain and intracellular (IC) domain of GP2. GP2 can be expressed without the IC domain if desired. Expression, for example, of GP1, GP2, NP and Z according to the methods described below would employ at least a tetracistronic construct, whereas one expressing GPC, NP and Z would employ one that is tricistronic. The GP1 signal peptide and GP2 transmembrane domain sequences can be substituted with like-functioning sequences in a heterologous manner. Certain versions of GP1 and GP2 proteins that can be employed in the instant invention are illustrated in FIGS. 1A-C of Mick et al. (2008, Virol J. 5:161; FIG. 1 thereof is herein incorporated by reference in its entirety).

The LASV NP protein employed for the instant invention may comprise or consist of SEQ ID NO:9. Alternatively, the NP protein may comprise or consist of an amino acid sequence that is at least about 70%, 75%, 80%, 85%, 90%, 91%, 92%, 93%, 94%, 95%, 96%, 97%, 98%, or 99% identical to SEQ ID NO:9. Such variants of SEQ ID NO:9 should function or behave (e.g., antibody binding activity, immunogenicity, arenaviral RNA binding) the same as or in a similar manner to SEQ ID NO:12 and/or other known NP proteins. Examples of NP proteins that can be used in the invention are disclosed at the U.S. NCBI website (or GenBank) under accession numbers NP_{—}694869, AAO59513, AAG41803, AAL13213 and AAF86704 (these sequences are herein incorporated by reference in their entirety). Skilled artisans will realize that DNA sequences can be used to express the NP protein component for practicing the invention. For example, a DNA sequence comprising or consisting of SEQ ID NO:10 may be used to express LASV NP protein. Alternatively, a DNA sequence comprising or consisting of a sequence that is at least about 60%, 70%, 75%, 80%, 85%, 90%, 91%, 92%, 93%, 94%, 95%, 96%, 97%, 98%, or 99% identical to SEQ ID NO:10 can be used to express LASV NP protein, just so long that the expressed product functions or behaves (e.g., antibody binding activity, immunogenicity, arenaviral RNA binding) the same as or in a similar manner to the NP protein. Examples of DNA sequences encoding NP proteins that can be used in the invention are disclosed at the U.S. NCBI website (or GenBank) under accession numbers AY628203 (positions 101-1810), J04324 (positions 101-1810), AY772168 (positions 1593-3302), AY179173 (positions 1573-3282), AY628205 (positions 97-1806) and AY628201 (positions 100-1809) (these sequences, particularly the regions therein that encode the NP proteins as shown parenthetically following each accession number, are herein incorporated by reference in their entirety).

Fragments of GPC (or GP1 and GP2 if expressed independently) and NP proteins can be expressed in the inventive Lassa VLPs, as none of these components is critical for VLP formation. It is well within the skill in the art to employ fragments by employing, for example, recombinant DNA techniques. Expression of fragments instead of full-length versions of the aforementioned proteins will permit testing the activity of specific sets of epitopes in different diagnostic and/or therapeutic regimes Fragments of these proteins may be expressed alone or in the form of a fusion protein, may have variations in amino acid sequence (refer to above percent identity values), and/or may contain inserted non-viral sequences. Finally, depending on which protein(s) is selected for fragment derivation, the fragments may be about 20, 40, 60, 80, 100, 120, 140, 160, 180, 200, 220, 240, 260, 280, 300, 320, 340, 360, 380, 400, 420, 440, 460, 480, 500, 520, 540, or 560 amino acids in length. These fragment lengths can be measured with respect to SEQ ID NOs:9 and/or 12, or other known versions of these proteins.

Functional analogs/variants/fragments of the VLP components (e.g., GPC, GP1, GP2, NP) can be those that function in the same or similar manner as a wildtype form of the component. Such function may be the ability to raise one or more antibodies to a native arenavirus from which the VLP component is derived or models. A short fragment (by itself or comprised within a larger sequence) of a component that is not necessary for VLP formation can be functional in this regard; e.g., it can serve to present one or more epitopes in an immunogenic method (e.g., vaccination) or a diagnostic method (e.g., ELISA).

Generally, the soluble forms of LASV GP1 and GP2 that can be used in the instant invention comprise all or part of the ectodomains of the native GP1 and GP2 protein subunits. Soluble forms of GP1 and GP2 can be produced by expressing GP1 and GP2 separately and deleting all or part of the transmembrane domain (TM) of the native mature LASV GP2 subunit protein and deleting all or part of the intracellular C-terminus domain (IC) of the native mature LASV GP2 subunit protein. By way of example, a soluble LASV GP2 glycoprotein may comprise the complete ectodomain of the native mature LASV GP2 glycoprotein.

The term ectodomain refers to that portion of a protein which is located on the outer surface of a cell (when expressed in context of a viral infection). For example, the ectodomain of a transmembrane protein is that portion(s) of the protein which extends from a cell's outer surface into the extracellular space (e.g., the extracellular domain of the mature native LASV GP2; refer to amino acids 260-427 of GPC). Further, an ectodomain can describe entire proteins that lack a transmembrane domain, but are located on the outer surface of a cell (e.g., mature native LASV GP1; refer to amino acids 59-259 of the GPC).

The methods of the instant invention include enhanced production techniques. This aspect of the invention preferably employs plasmid constructs (i.e., expression vectors) encoding the VLP components described above in a bicistronic or tricistronic manner; however, tetracistronic, pentacistronic, hexacistronic, heptacistronic and other multicistronic constructs are envisioned. A preferred embodiment of the instant invention comprises an expression vector that contains individual expression cassettes (one cassette minimally contains a promoter and ORF) for LASV Z, GPC and NP proteins, respectively (exemplifies a tricistronic vector). The ORFs in the inventive constructs can be in any order with respect to each other. Such constructs can utilize the same or different promoters to drive expression of each ORF (open reading frame). As described above, the LASV components can be fused to other proteins or can contain variable sequences with respect to previously known LASV sequences. Other preferred embodiments constitute bicistronic vectors that comprise LASV Z+GPC or Z+NP cassette combinations (any order). By “enhanced” production, it is meant that the VLP components and/or VLPs themselves of the instant invention are expressed in a manner that is at least 10%, 25%, 50%, 75%, 100%, 125%, 150%, 175%, 200%, 225%, 250%, 300%, 400%, 500% or 1000% greater than the same components or VLPs produced according to previously known methods.

It is also possible to practice the invention using IRES (internal ribosomal entry site) sequences, which allow for the production of multicistronic constructs that drive expression of more than one ORF from the same promoter. Thus, if employing IRES sequences, one could express LASV Z+GPC+NP (any order) from the same construct under one promoter (using two IRES sequences) or two promoters (two cassettes, one with one ORF, the other with two ORFs having one IRES sequence), for example. IRES sequences are well known in the art; for example, the IRES from encephalomyocarditis virus (EMCV) is applicable for practicing the instant invention. This IRES permits protein expression in both eukaryotic cells and cell-free extracts.

Plasmids are preferred for preparing the expression constructs of the present invention. However, other vector types can be employed if desired. “Vector” as used herein refers to any DNA molecule used as a vehicle to transfer foreign genetic material into a cell. The four major types of vectors applicable to the invention are plasmids, viruses (e.g., retrovirus, adenovirus, AAV), cosmids, and artificial chromosomes (e.g., bacterial artificial chromosomes). All of these vector types can be used in cells in either an episomal state (e.g., how a vector might exist in a transient expression system) or stable state (i.e., where the vector has integrated into a chromosome of a cell). Introduction of expression vectors/constructs can be performed by any number of protocols known in the art, such as transfection or transduction.

Multicistronic vectors can also contain cassettes (or follow an IRES) for expressing non-LASV proteins. Such proteins can be selected, for example, for tagging or marking the expressed VLPs for ease of detection and/or purification, or for rendering VLPs more immunogenic (i.e., an adjuvant protein). These “auxiliary” proteins are well known to skilled artisans.

Promoters suitable for driving one or more of the above-described proteins of the inventive VLPs are well known to skilled artisans. For example, the promoter(s) may be selected from those that are constitutive (e.g., from a housekeeping gene or viral gene), inducible, tissue- or cell-specific. Examples of promoters that can be used in the instant invention are the SV40 promoter, CMV promoter, adenovirus major late promoter, Rous sarcoma virus promoter, beta-actin promoter, MMTV promoter, and Mo-MLV promoter.

“Recombinant expression cassettes”, “expression cassettes”, “expression constructs”, expression vectors” for use in the instant invention can be a nucleic acid construct, generated recombinantly or synthetically, that have control elements capable of effecting expression of a structural gene that is operatively linked to the control elements in hosts compatible with such sequences. Expression cassettes include at least promoters and optionally, transcription termination signals. Typically, the recombinant expression cassette includes at least a nucleic acid to be transcribed (e.g., a nucleic acid encoding a desired polypeptide) and a promoter. Additional factors necessary or helpful in effecting expression can also be used as described herein. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette.

The DNA sequences used to practice the invention may be analyzed and prepared according to practices well known in the art. For example, gene synthesis may be accomplished by standard cloning techniques or via in silico and/or artificial/chemical means. Methods of artificial/chemical production of large genetic sequences have been described, for example, by Abhishek (2009, Efficient in silico Designing of Oligonucleotides for Artificial Gene Synthesis, Nature Protocols 10.1038/nprot.2009.15) and in U.S. Pat. No. 6,521,427, both of which references are herein incorporated by reference in their entirety.

In general, VLPs can be produced by introducing into a cell a vector (e.g., tricistronic vector) as described herein. This is in contrast to previous attempts by others in which separate vectors were used to express each VLP component. However, the instant invention is also drawn to practices wherein an additional vector(s), for example one expressing an auxiliary or adjuvant protein, is introduced separately from the multicistronic viral protein-encoding vector. The viral proteins are translated/expressed and self-assembled into a VLP. The cells can include, but are not limited to, insect cells (e.g., Spodoptera frugiperda Sf9 cells and Sf2 cells) and mammalian cells (e.g., EL4, HeLa, HEK-293, VERO, BHK).

The inventive VLPs can be expressed in vivo in mammalian cells, yeast cells, Xenopus eggs and insect cells (e.g., using a baculovirus expression system), for example. Expression can also be performed in vitro using cell-free extracts.

While VLPs of the invention closely resemble mature virions, they generally do not contain viral genomic material (i.e., RNA). Therefore, the inventive VLPs are non- replicative in nature, which make them safe for administration in the form of an immunogenic composition (e.g., vaccine). In addition, the inventive VLPs can express envelope glycoproteins on the surface thereof, which is the most physiological configuration; this better ensures that an immune response against a VLP-based vaccine will block or inhibit an infection by actual virus. Indeed, since the inventive VLPs resemble intact virions and are multivalent in structure (e.g., exhibit multiple epitopes), they can be more effective in inducing neutralizing antibodies to viral components compared to the use of soluble antigens typically used in a vaccine. Further, the VLPs of the instant invention can be administered repeatedly to vaccinated hosts, unlike many recombinant vaccine approaches.

One embodiment of the instant invention is drawn to VLPs comprising an arenavirus matrix protein (Z), an arenavirus glycoprotein (GP1 and/or GP2 as processed from an arenavirus GPC) and an arenavirus nuclear protein (NP). In addition, the VLP can include at least one adjuvant molecule. Another embodiment comprises a matrix protein (Z) and a nuclear protein (NP). VLPs of the invention can contain any other arenavirus gene product, whether it be a structural protein or and enzymatic protein. Furthermore, the VLP can include a lipid membrane. The VLPs of the instant invention can be chimeric in that they may contain protein components from more than one arenavirus, or can be comprised predominantly with arenaviral components but include components from other virus types.

Examples of adjuvant molecules that can be incorporated in the inventive VLPs are the VEE (Venezuelan equine encephalitis) adjuvant molecule, Flt3, the mannose adjuvant molecule, CD40, and C3d. In particular, VEE, the Flt3, the mannose adjuvant molecule, and CD40 can be used to target dendritic cells, while C3d can be used to target follicular dendritic cells. Mannose molecules can be chemically added to VLPs after the VLPs are produced.

The present invention also includes an immunogenic composition. The immunogenic composition includes a pharmacologically acceptable carrier and at least one of the VLPs described herein. Further, another embodiment of the present invention includes a method of generating an immunological response in a host by administering an effective amount of one or more of the immunogenic compositions described herein to the host. The present invention includes a method of treating a condition by administering to a host in need of treatment an effective amount of one or more of the immunogenic compositions described herein.

The above immunogenic compositions can be used particularly to enhance immune responses such as antibody production (humoral response), cytotoxic T cell activity (cellular response) and cytokine activity. In this regard, VLPs can be administered prophylactically in a vaccine program to prevent viral infections caused by the arenavirus for which the VLP models or is acting as a surrogate.

Immunogenic compositions can relate to vaccines for LASV and other arenaviruses. In one aspect, the vaccine comprises VLPs. In another aspect, the vaccine is a DNA-based vaccine in which one of the above-described vectors is administered for VLP expression in vivo. Administration of expression vectors includes local or systemic administration, including injection, oral administration, particle gun or catheterized administration, and topical administration. Targeted delivery of therapeutic compositions containing an expression vector or subgenomic polynucleotides can also be used. For human administration, the codons comprising the polynucleotide encoding one or more VLP components may be optimized for human use, a process which is standard in the art.

Embodiments of the invention can be directed to methods of enhancing or increasing the immunogenicity of an LASV VLP by co-expressing NP with Z and GPC (or with Z, GP1, and GP2. For example, such a method could be performed by first preparing VLPs using an expression construct having sequences encoding NP and other LASV proteins such as Z and GPC.

Examples of antibodies encompassed by the present invention, include, but are not limited to, antibodies specific for proteins of the inventive VLPs, antibodies that cross react with native LASV antigens, and neutralizing antibodies. By way of example a characteristic of a neutralizing antibody includes the ability to block or prevent infection of a host cell. The antibodies of the invention may be characterized using methods well known in the art.

The antibodies useful in the present invention can encompass monoclonal antibodies, polyclonal antibodies, antibody fragments (e.g., Fab, Fab', F(ab')2, Fv, Fc, etc.), chimeric antibodies, bi-specific antibodies, heteroconjugate antibodies, single-chain fragments (e.g., ScFv), mutants thereof, fusion proteins comprising an antibody portion, humanized antibodies, and any other modified configuration of the immunoglobulin molecule that comprises an antigen recognition site of the required specificity, including glycosylation variants of antibodies, amino acid sequence variants of antibodies, and covalently modified antibodies. The antibodies may be murine, rat, human, or of any other origin (including chimeric or humanized antibodies).

Methods of preparing monoclonal and polyclonal antibodies are well known in the art. Polyclonal antibodies can be raised against the inventive VLPs in a mammal, for example, by one or more injections of an immunizing agent and, if desired an adjuvant. Examples of adjuvants include, but are not limited to, keyhole limpet hemocyanin (KLH), serum albumin, bovine thryoglobulin, soybean trypsin inhibitor, complete Freund adjuvant (CFA), and MPL-TDM adjuvant. hi other embodiments, vaccines are provided that comprise VLPs, but without an adjuvant. Examples of vaccines that do not comprise an adjuvant can include those with VLPs comprising Z+GPC and Z+GPC+NP.

The antibodies may alternatively be monoclonal antibodies. Monoclonal antibodies may be produced using hybridoma methods (see, e.g., Kohler, B. and Milstein, C. (1975) Nature 256:495-497 or as modified by Buck, D. W., et al., In Vitro, 18:377-381(1982). In another alternative embodiment of the invention, antibodies may be made recombinantly and expressed using any method known in the art. By way of example, antibodies may be made recombinantly by phage display technology. See, for example, U.S. Pat. Nos. 5,565,332; 5,580,717; 5,733,743; and 6,265,150 (all these patents are herein incorporated by reference in their entirety).

The present invention is also directed to medicaments containing the VLP compositions described herein. Also, use of the inventive VLPs for the manufacture of a medicament represents an aspect of the invention. It should be understood that certain compositions of the present invention may comprise multiple components such as an appropriate pharmaceutical carrier, diluent, or excipient. Various pharmaceutical carriers and other components for formulating the peptide for therapeutic use are described in U.S. Pat. Nos. 6,492,326 and 6,974,799, both of which are incorporated herein by reference in their entirety.

Another embodiment of the present invention includes methods of determining exposure of a host to a virus. An exemplary method, among others, includes the steps of: contacting a biological fluid of a host with one or more of the VLPs discussed above, wherein the VLP is of the same virus type to which exposure is being determined, under conditions which are permissive for binding of antibodies in the biological fluid with the VLP; and detecting binding of antibodies within the biological fluid with the VLP, whereby exposure of the host to the virus is determined by the detection of antibodies bound to the VLP. Skilled artisans will recognize that this methodology is amenable to practicing an enzyme-linked immunosorbant assay (ELISA) and assays involving lateral flow strips (movement by capillary action).

The term “host” includes mammals (e.g., humans, cats, dogs, horses, and cattle), and other living species that are in need of treatment. Hosts that are “predisposed to” condition(s) can be defined as hosts that do not exhibit overt symptoms of one or more of these conditions but that are genetically, physiologically, or otherwise at risk of developing one or more of these conditions. The terms “treat”, “treating”, and “treatment” together represent an approach for obtaining beneficial or desired clinical results. For purposes of embodiments of this invention, beneficial or desired clinical results include, but are not limited to, alleviation of symptoms, diminishment of extent of disease, stabilization (i.e., not worsening) of disease, preventing spread of disease, delaying or slowing of disease progression, amelioration or palliation of the disease state, and remission (partial or total) whether detectable or undetectable.

By an “effective” amount (or “therapeutically effective” amount) of a pharmaceutical composition is meant a sufficient, but non-toxic amount of the agent to provide the desired effect. The term refers to an amount sufficient to treat a subject. Thus, the term therapeutic amount refers to an amount sufficient to remedy a disease state or symptoms, by preventing, hindering, retarding or reversing the progression of the disease or any other undesirable symptoms whatsoever. The term prophylactically effective amount refers to an amount given to a subject that does not yet have the disease, and thus is an amount effective to prevent, hinder or retard the onset of a disease.

Modifications may occur anywhere in the polypeptides of the present invention, including the peptide backbone, the amino acid side-chains and the amino- or carboxy-termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched, and branched cyclic polypeptides may result from post-translation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

“Variants” refers to polypeptides of the present invention that differ from a reference polynucleotide or polypeptide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, and deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

“Identity” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including, but not limited to, those described in Computational Molecular Biology, Lesk, A. M., Ed., Oxford University Press, New York, (1988); Biocomputing : Informatics and Genome Projects, Smith, D. W., Ed., Academic Press, New York, (1993); Computer Analysis of Sequence Data, Part I, Griffin, A. M., and Griffin, H. G., Eds., Humana Press, New Jersey, (1994); Sequence Analysis in Molecular Biology, von Heinje, G. Academic Press, 1987; and Sequence Analysis Primer, Gribskov, M. and Devereux, J., Eds., M Stockton Press, New York, (1991); and Carillo, H., and Lipman, D., SIAM J Applied Math., 48,1073 (1988) (all these references are herein incorporated by reference in their entirety).

In general, homologous polypeptides of the present invention are characterized as having one or more amino acid substitutions, deletions, and/or additions. These changes are preferably of a minor nature (e.g., conservative amino acid substitutions and other substitutions that do not significantly affect the activity of the polypeptide). Among the common amino acids, for example, a conservative amino acid substitution is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. Other conservative amino acid substitutions include amino acids having characteristics such as a basic pH (arginine, lysine, and histidine), an acidic pH (glutamic acid and aspartic acid), polar (glutamine and asparagine), hydrophobic (leucine, isoleucine, and valine), aromatic (phenylalanine, tryptophan, and tyrosine), and small (glycine, alanine, serine, threonine, and methionine).

All of the embodiments of the inventive compositions may be in the “isolated” state. For example, an “isolated” nucleic acid molecule, as used herein, is one that is separated from nucleic acids which normally flank the gene or nucleotide sequence and/or has been completely or partially purified. As another example, an isolated composition of the invention (e.g., VLP) may be substantially isolated with respect to the complex physiological milieu in which it naturally occurs. In some instances, the isolated composition will be part of a greater composition (e.g., a crude extract containing other substances), buffer system or reagent mix. In other circumstances, the inventive composition may be purified to essential homogeneity. An isolated composition may comprise at least about 50, 80, 90, or 95% (on a molar basis) of all the other macromolecular species that are also present therein. The VLPs of the instant invention do not exist naturally; the instant invention does not embrace, for example, aberrant viral particles that are shed from an infected cell that might be deficient in one or more normal nucleic acid and/or protein components. The nucleic acids of the instant invention (e.g., constructs, vectors) do not exist naturally and do not embrace viral genome sequences as they might exist in an unmodified state. The inventive compositions may comprise heterologous combinations of components. For example, a protein-coding region of a viral gene may be driven by a promoter not derived from the gene. The same rationale applies to other gene expression components, such as terminator sequences and introns (e.g., first intron). For example, the constructs can comprise non-LASV promoter(s) and/or terminator sequences. The inventive nucleic acid constructs are not infectious (i.e., they cannot produce a fully functional virus), such as the case of an infectious cDNA, which generally comprises viral regulatory (e.g., repeat regions, origin or replication) and replicative (polymerases) sequences. While preferred embodiments of the inventive VLPs do not contain nucleic acid sequences (e.g., viral genomic sequence), other embodiments may be engineered to contain at least one heterologous sequence.

An embodiment of the invention is directed to the early diagnosis of LASV infection. This embodiment can be performed by detecting GP1 in the blood, serum, or any other fluid or tissue of an individual that is non part of the reticuloendothelial system, but without likewise detecting other LASV components such as GPC, GP2, NP and/or Z proteins. Such GP1 is soluble GP1 (sGP1), as it is not associated with virion particles. Detection of GP1 in this embodiment can be associated with an infection that has occurred within about the past 0, 1, 2, 3, 4, 5, 6, 7, 8, or 9 days. Alternatively, GP1 detection can be made before the onset of febrile disease, or before antibodies to one or more LASV proteins can be detected in an infected individual.

The following examples are included to demonstrate certain preferred embodiments of the invention for extra guidance purposes. As such, these examples should not be construed to limit the invention in any mariner.

### EXAMPLES

### Example 1

Cloning of the LASV gene encoding Z matrix protein.

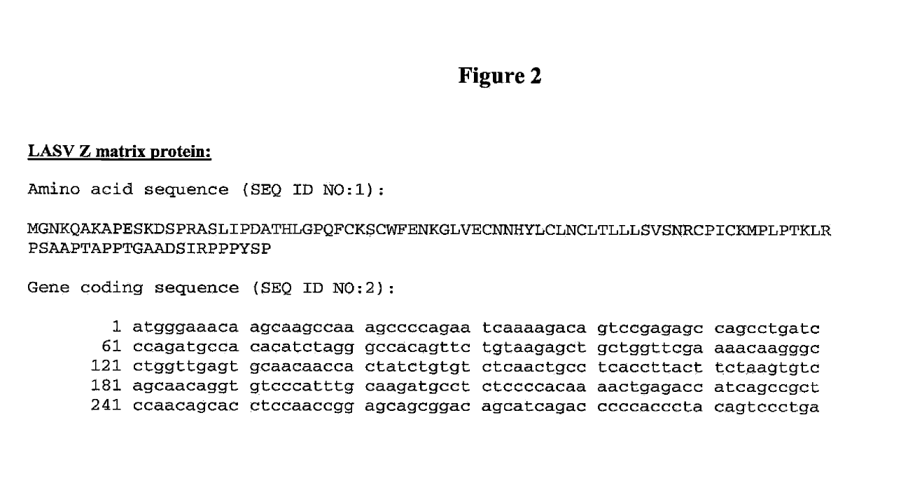

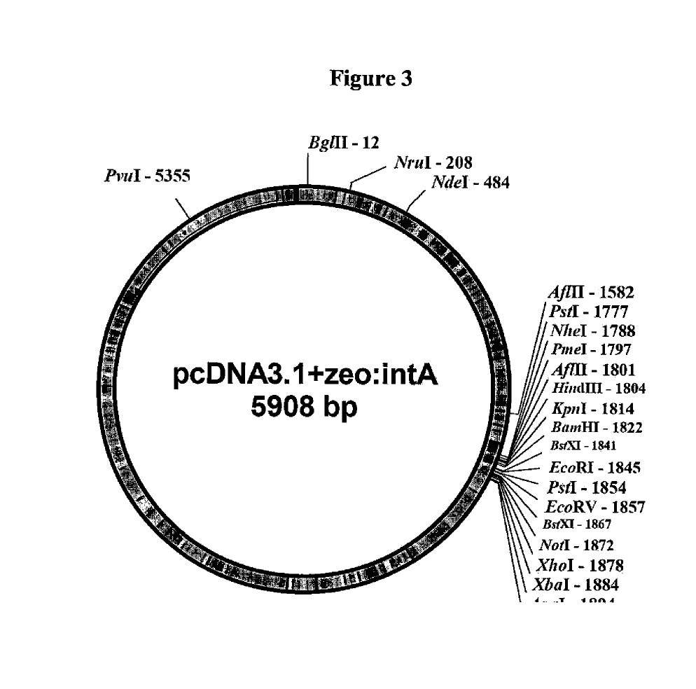

The 99 amino acid LASV Z matrix protein gene was amplified from total RNA isolated from Lassa virus Josiah strain-infected Vero cells at six days post infection. FIG. 2 shows the. Josiah strain Z protein amino acid sequence (NCBI Accession no. AAT49001) and corresponding encoding DNA. Infected cells were collected from culture dishes and dissolved in Trizol® reagent (Invitrogen, Carlsbad, Calif.). Total RNA was extracted from Trizol® suspensions as per the manufacturer's instructions. RNA was resuspended in DEPC-treated water and stored at −80° C. One microgram of total RNA was reverse transcribed to complementary DNA (cDNA) using Invitrogen's SuperScript® II system. cDNAs were subjected to polymerase chain reaction (PCR) with gene-specific primers and amplified with Phusion® High Fidelity DNA Polymerase (New England Biolabs, Ipswich, Mass.). Amplification of gene products was confirmed by agarose gel electrophoresis, followed by cloning into pTOPO Zero Blunt-II® vectors (Invitrogen), or by digestion with restriction endonucleases (RENs) specific for sites engineered at the 5′ and 3′ ends of each gene construct and directly cloning into the modified mammalian expression vector pcDNA3.1+zeo:intA (intronless pcDNA3.1+zeo from Invitrogen) (FIG. 3). The pcDNA3.1+zeo:intA contains the HCMV intronA sequence downstream of the basic CMV promoter, which drives heterologous gene expression. Expression constructs were subjected to double-stranded DNA sequencing and REN digestion for verification of sequence accuracy and gene orientation. One bacterial clone harboring the verified construct was expanded for cryopreservation and large scale purification of plasmid DNA.

The following primers were used as above to amplify the LASV Z matrix DNA coding sequence. SEQ ID NO:5 was used as the 3′ primer when adding a His-tag epitope coding sequence to the carboxy terminus of the Z protein.

5′ Z protein oligo (HindIII site and kozak sequences underlined):
(SEQ ID NO: 3)
cagtaagcttccaccatgggaaacaagcaagccaaagccccagaa
3′ Z protein oligo (NotI site and two ectopic stop codons:
[opposite sense] underlined)
(SEQ ID NO: 4)
actggcggccgctcagtcatcagggactgtagggtgggggtctgatgct
3′ Z protein oligo (His-tag sequence, NotI site and two
ectopic stop codons [opposite sense] underlined):
(SEQ ID NO: 5)
actggcggccgctcagtcagtgatggtgatggtgatggggactgtagggtgggggtc
tgatgct

### Example 2

Generation of bicistronic and tricistronic vectors for high level expression of LASV VLP.

A tricistronic vector for the expression of LASV GPC, NP, and Z genes from one locus was engineered by using a single gene construct as a backbone. A modified pcDNA3.1+zeo:intA vector was used for high level expression of LASV genes in mammalian cells. In building the constructs used in the below-discussed expression studies, a pcDNA3.1+zeo:intA construct already containing the GPC, NP or Z gene sequence served as a backbone for further introduction of one or two other LASV sequences. For this example pcDNA3.1+zeo:intA:LASV GPC was used as the initial construct into which additional expression cassettes (i.e., NP and/or Z genes) were placed; the second expression cassette was inserted at the unique NruI site located upstream of the 5′ end of CMV promoter. In this case, a LASV NP expression cassette containing the complete CMV promoter, intronA sequence, the Kozak sequence-optimized LASV NP open reading frame (ORF), and a BGHpA (BGH polyA sequence signal), flanked by NruI sites was PCR-amplified from pcDNA3.1+zeo:intA:LASV NP and cloned into the unique NruI site in pcDNA3.1+zeo:intA:LASV GPC. A similar approach was then employed to PCR-amplify the LASV Z cassette flanked by a complete CMV promoter and BGHpA, but containing BglII ends. The cassette was then cloned into the unique BglII site in the pcDNA3.1+zeo:intA:LASV GPC+LASV NP construct, thereby generating pcDNA3.1+zeo:intA:LASV_GPC+NP+Z (an example of a tricistronic vector of the instant invention).

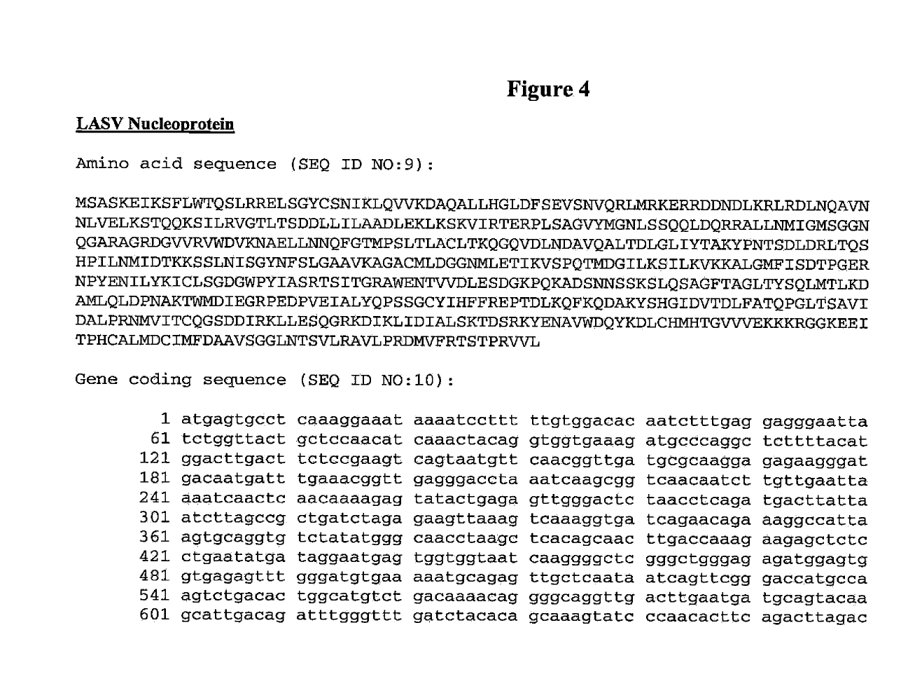

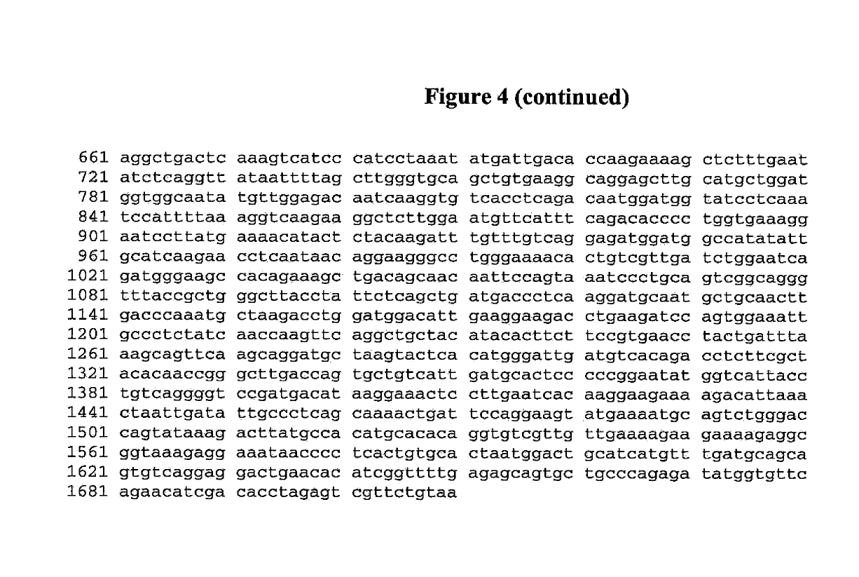

The following primers were used to amplify the LASV NP (nucleoprotein) DNA coding sequence as part of the construct building process. FIG. 4 shows the Josiah strain NP protein amino acid sequence (NCBI Accession no. NP_{—}694869) and corresponding encoding DNA.

5′ NP oligo (HindIII site and kozak sequences
underlined):
(SEQ ID NO: 6)
cagtaagcttccaccatgagtgcctcaaaggaaataaaatcctttttg
3′ NP oligo (NotI site and two ectopic stop codons
[opposite sense] underlined):
(SEQ ID NO: 7)
actggcggccgctcagtcacagaacgactctaggtgtcgatgt

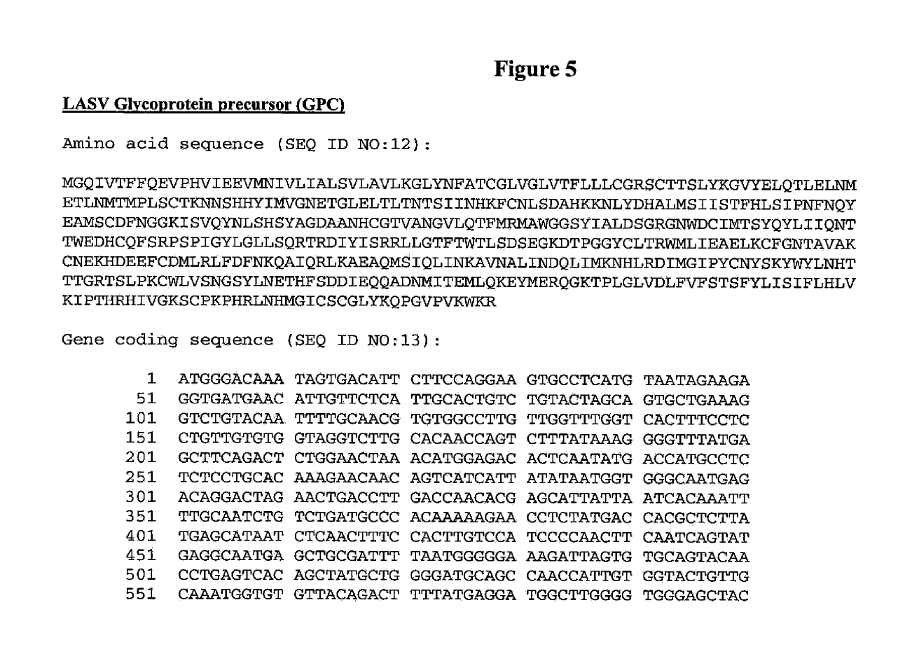

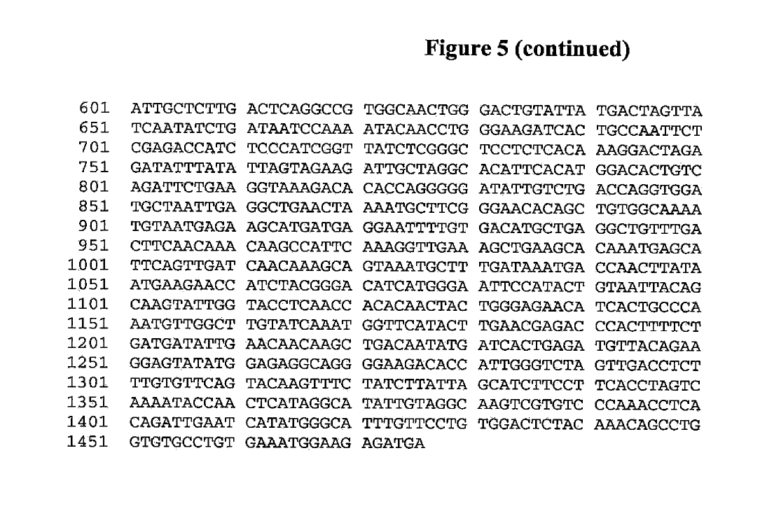

The following primers were used to amplify the LASV GPC DNA coding sequence as part of the construct building process. FIG. 5 shows the Josiah strain GPC protein amino acid sequence (see NCBI Accession no. NP_{—}694870) and corresponding encoding DNA.

5′ GPC oligo (NheI site underlined):
(SEQ ID NO: 10)
gtagctagcatgggacaaatagtgacattcttccag
3′ GPC oligo (HindIII site and two stop codons
[opposite sense] underlined):
(SEQ ID NO: 11)
ggtaccaagctttcagtcatctcttccatttcacaggcac

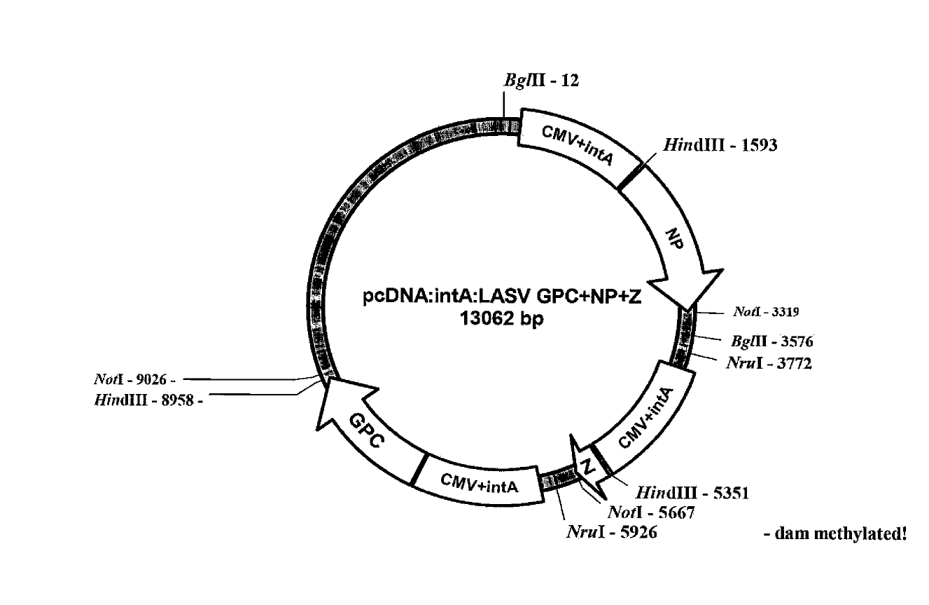

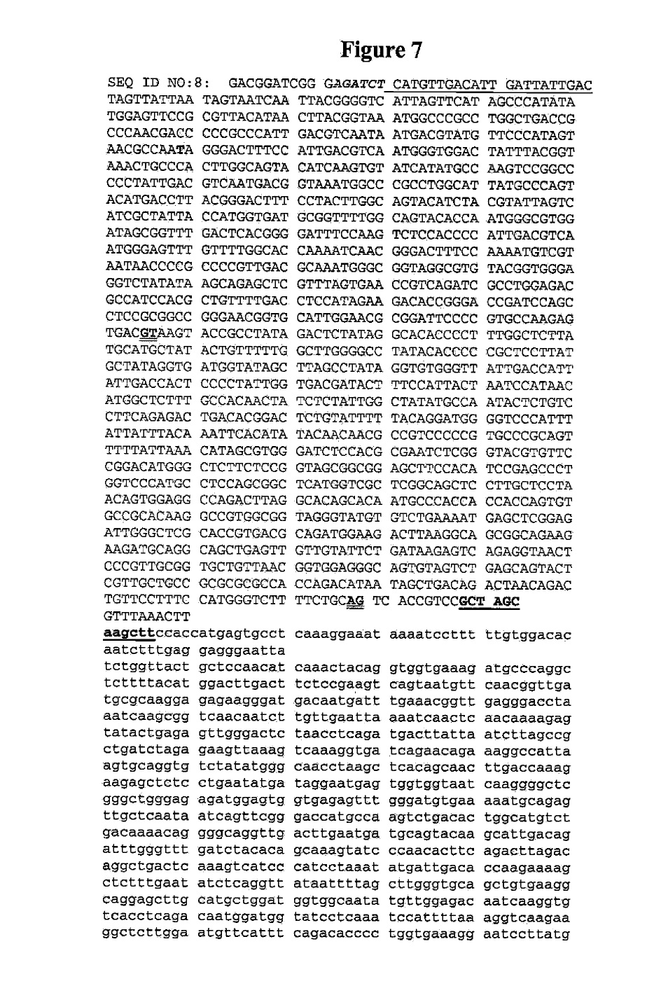

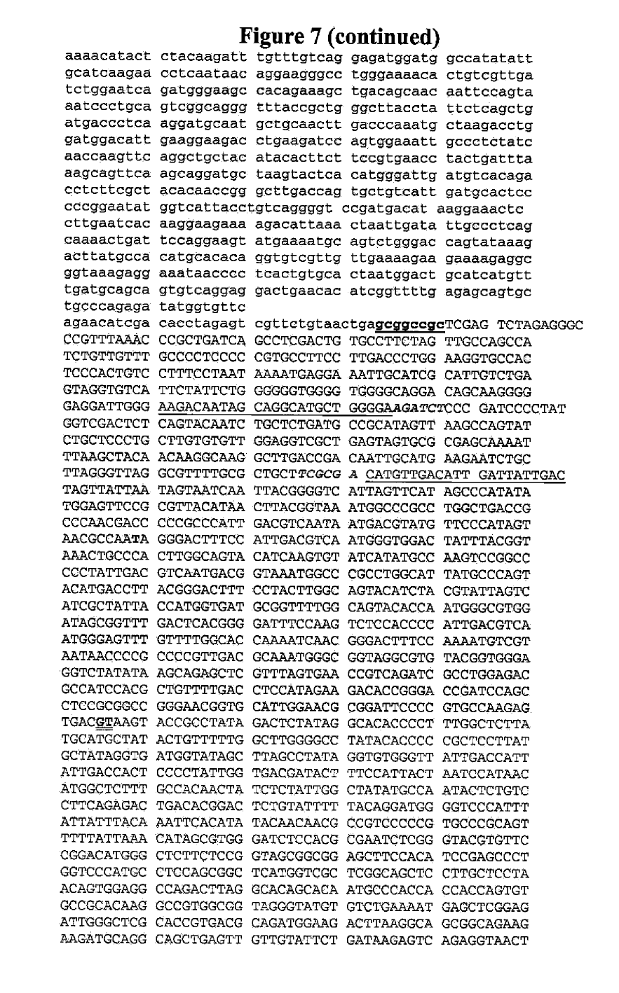

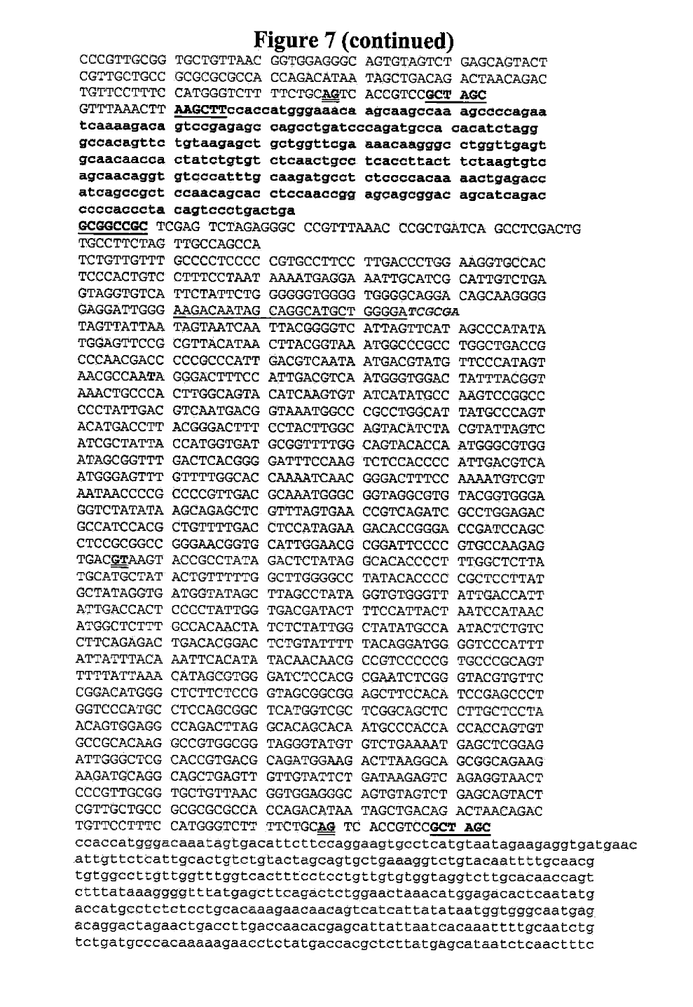

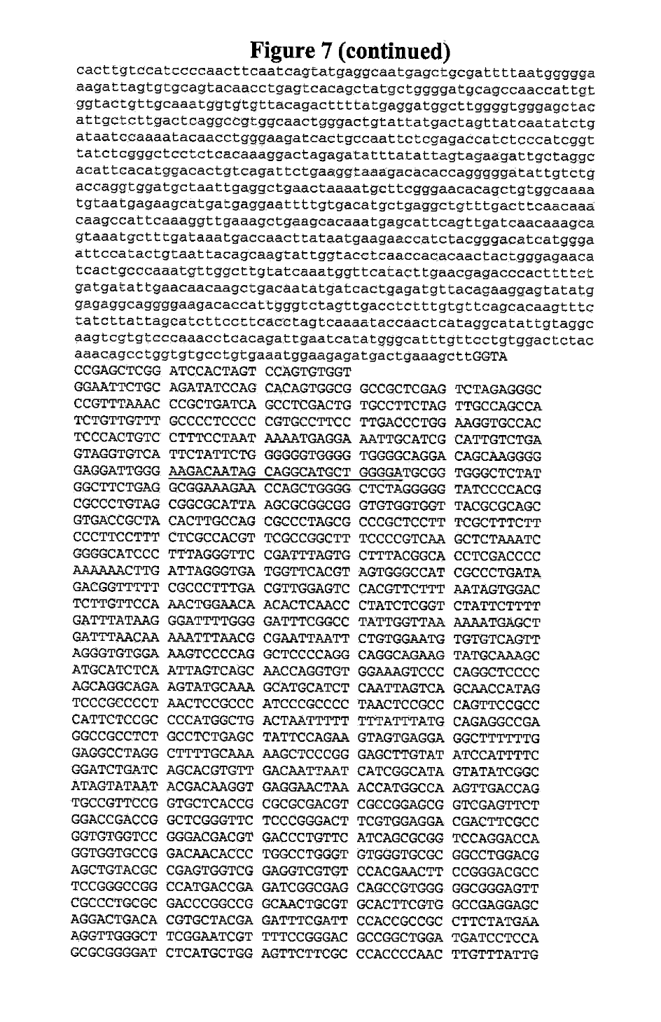

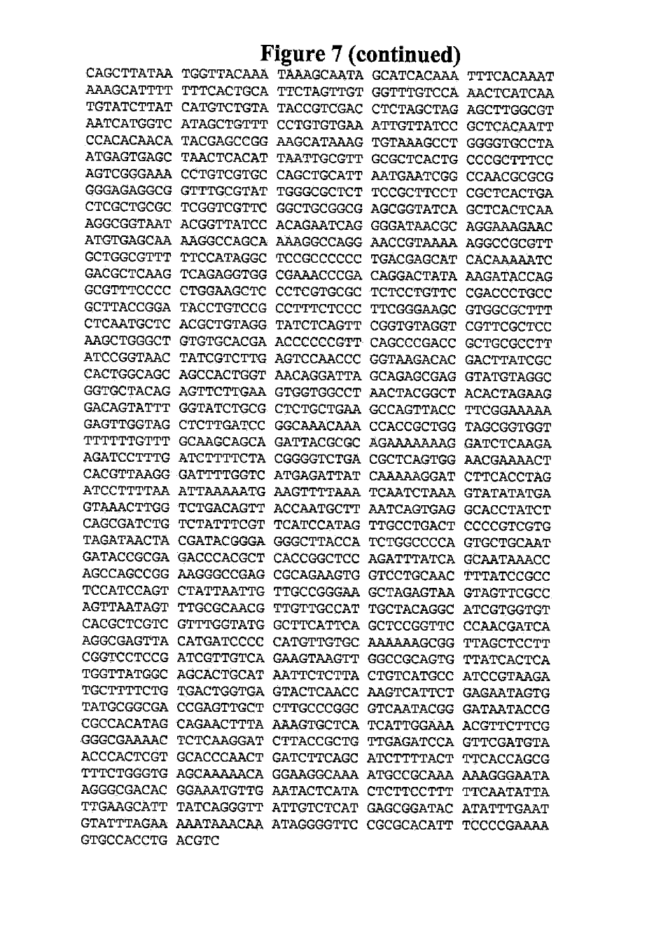

The pcDNA3.1+zeo:intA:LASV_GPC+NP+Z construct prepared as above is depicted in FIG. 6. A complete sequence of this construct is rendered in FIG. 7 following the same basepair numbering scheme as shown in FIG. 6. Pertinent restriction and primer binding sites that are discussed above are emphasized visually (e.g., underlining) in the sequence.

### Example 3

Expression of Lassa VLPs in mammalian cells.

Transient expression of LASV gene constructs

Recombinant LASV protein expression was analyzed in HEK-293T/17 cells transiently transfected with mammalian expression vectors, which were prepared using the PureLink® HiPure plasmid filter midiprep system (Invitrogen). The negative control vector pcDNA3.1(+):intA was included in all transfections. Briefly, 1×10⁶ cells were seeded per well of a poly-D-lysine-coated 6-well plate in 2 mL of Complete Dulbecco's minimal essential medium (cDMEM). After overnight incubation under standard growth conditions (37° C., 5% CO₂, 90% RH), cells were transfected with unrestricted (i.e., non-linearized) control and recombinant plasmid DNAs using Lipofectamine™ 2000 (Invitrogen), according to the manufacturer's instructions. Four μg of each plasmid DNA were used per transfection.

Transfections were incubated for the times described below under standard growth conditions after which cell culture supernatants were collected and clarified by centrifugation; depending on what LASV proteins were expressed, these supernatants may contain VLPs. To prepare cell extracts from transfected cultures, cell monolayers were carefully washed twice with Ca⁺⁺- and Mg⁺⁺-free PBS, pH 7.4, collected by gentle dislodging, transferred to 1.5-mL polypropylene tubes, and lysed for 10 minutes in a mammalian cell lysis buffer comprised of 50 mM Tris buffer, pH 7.5, 1 mM EDTA, 0.1% SDS, 0.5% deoxycholic acid, 1% Igepal® CA-360, and a protease inhibitor cocktail (Sigma Aldrich, St. Louis, MO), according to the manufacturer's instructions. The insoluble fraction was pelleted by centrifugation at 14,000× g for 10 minutes, and the supernatants were transferred to fresh tubes. The protein concentration of each sample was determined by A280 with A260 subtraction, and verified using a Micro BCA™ Protein Assay Kit, as outlined by the manufacturer (Thermo Scientific, Rockford, Ill.).

Generation, purification, and protein content assay of LASV VLPs

LASV VLPs were generated either at a small scale level in 6-well cell culture plates, or at a larger scale level in 15-cm culture dishes. For small scale generation of VLP, HEK-293T/17 cells were transfected with plasmid DNAs as described above and incubated for 72 hours prior to harvesting culture supernatants. Transfections in 15-cm culture dishes were scaled linearly and were likewise harvested at 72 hours.

Cell supernatants were cleared by low speed centrifugation (200× g, 5 minutes, at room temperature in a swinging bucket rotor). Polyethylene glycol-6000 (PEG-6000) and sodium chloride (NaCl) were mixed with the cleared supernatants to final concentrations of 5% and 0.25M, respectively. The reactions were incubated at 4° C. overnight, followed by centrifugation in an SW28 rotor at 15,000× g, 4° C., for 1 hour in a Sorvall® ultracentrifuge to pellet the VLP. Pellets from individual tubes were resuspended in 0.5 mL TNE buffer (20 mM Tris-base, 0.1M NaCl, 0.1 mM EDTA, pH 7.4) (or PBS), overlaid on a 3.5-mL 20% sucrose cushion (0.875 mL of each of 20%, 30%, 40% and 60% sucrose solutions were overlaid top-to-bottom, respectively, without mixing), and centrifuged in an SW60Ti rotor at 55,000 RPM, 4° C., for 2.5 hours in a Sorvall® ultracentrifuge to pellet the VLP. The VLP pellet was gently resuspended in the appropriate volume of TNE for analysis. VLP for immunizations were further purified through 20-60% discontinuous sucrose gradients, by ultracentrifugation, as outlined above.

Protein concentration was determined for each VLP sample by A280 with A260 subtraction, and verified using a Micro BCA™ Protein Assay Kit, as outlined by the manufacturer, using a Bovine Serum Albumin (BSA) standard curve (Thermo Scientific).

Western Blot and Densitometry Analyses

Expression of LASV glycoproteins in cell extracts and VLPs was confirmed by Western blot analysis using anti-LASV GP1-specific mAbs and a horseradish peroxidase (HRP)-conjugated goat anti-mouse IgG (H+L) secondary antibody. Likewise, expression of 6X-HIS-tagged Z matrix protein was confirmed with a mouse anti-HIS mAb (Invitrogen) and an HRP-conjugated goat anti-mouse IgG (H+L) secondary antibody. Expression of LASV NP was confirmed with affinity-purified goat IgG fraction raised against LASV NP and an HRP-conjugated rabbit anti-goat IgG (H+L) secondary antibody. Briefly, 10 μg of total cell protein in 10 μL (1×10⁵ cell equivalents) was resolved on 10% NuPAGE® Novex® Bis-Tris gels, according to the manufacturer's specifications (Novex, San Diego, Calif.). For analysis of VLPs, 1-10 μg of total protein (obtained after sucrose gradient centrifugation) as determined by A280 and BCA analyses was similarly resolved by SDS-PAGE. All samples in these studies were denatured and reduced in SDS-PAGE buffer containing DTT. Proteins were transferred to 0.45-μm nitrocellulose membranes, blocked, and probed in 1× PBS, pH 7.4, 5% non-fat dry milk, 0.05% Tween®-20, and 0.1% thymerosal. Membranes were then incubated in LumiGLO® chemiluminescent substrate (KPL, Gaithersburg, Md.) and exposed to Kodak® BioMax™ MS Film. Developed films were subjected to high resolution scanning for densitometry analysis. Quantification of band intensity was performed using National Institutes of Health ImageJ 1.41o software (available at rsb.info.nih.gov/ij website), and following the procedure outlined on the website lukemiller.org/journal/2007/08/quantifying-western-blots-without using TIFF files.

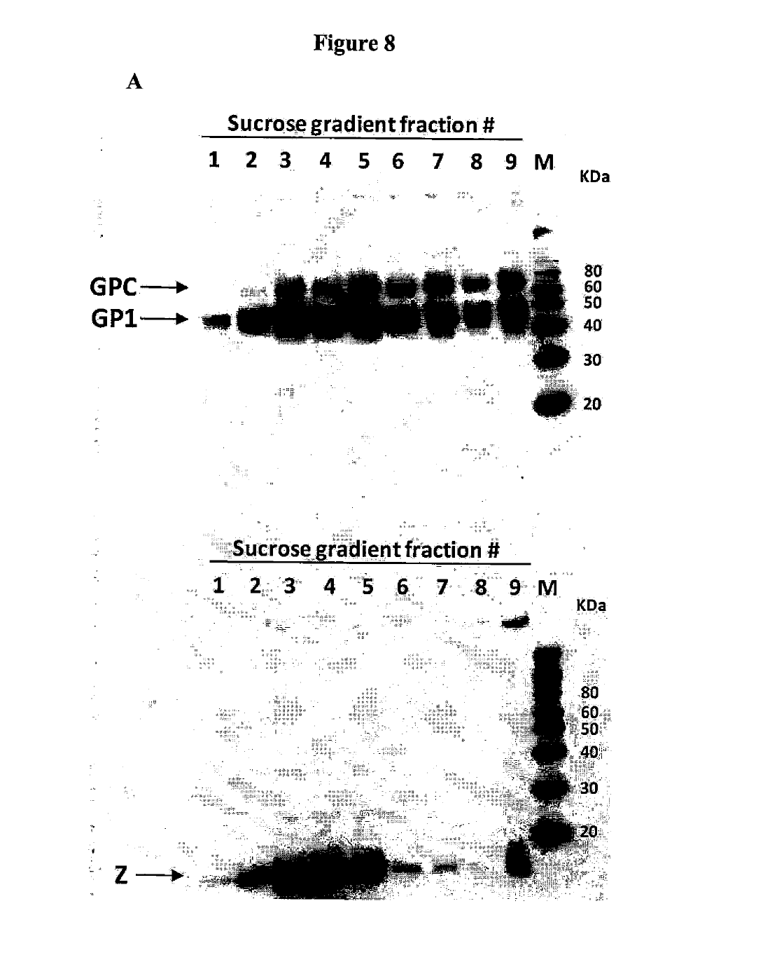

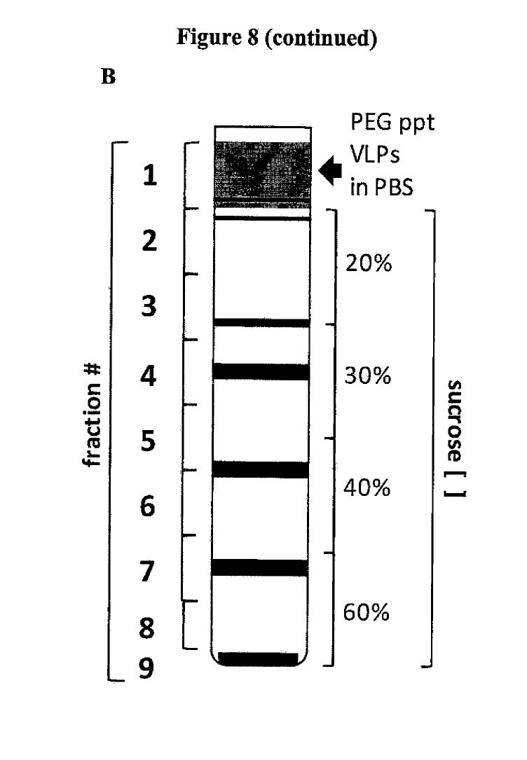

FIG. 8A shows the western blot analysis of sucrose gradient fractions collected as discussed above. Proteins were detected using either anti-LASV GP1 mAb (1:1000 dilution) (top blot) or anti-His tag (1 μg/mL), which detects His-tagged LASV Z protein (bottom blot). FIG. 8B depicts the sucrose pelleting results diagrammatically and from where each fraction of the sucrose gradient was taken for gel loading purposes. About 1000 μL of each fraction (1-8) was obtained from two identical sucrose cushions; fraction 9 (from two cushions), which represented mostly insoluble material, was resuspended in a total volume of 1000 μL 60% sucrose. Ten μL of each isolated fraction was resolved on each gel.

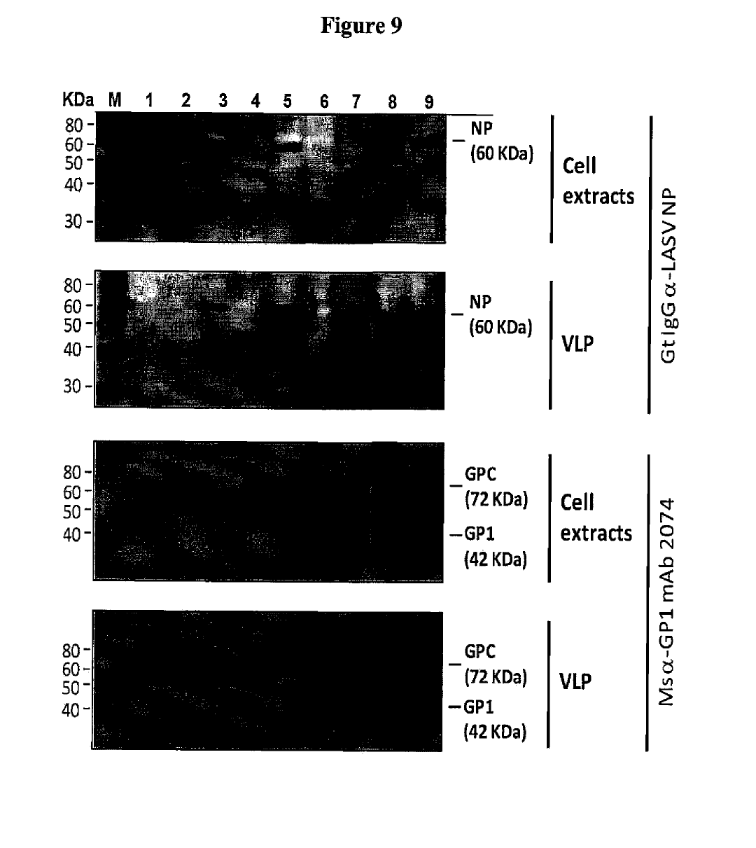

FIG. 9 shows the western blot analysis of LASV proteins as detected in cell extracts (first and third blots) and in isolated VLPs (second and fourth blots). The constructs used to achieve this expression were as follows:

| | |
|---|---|
| Lane 1: LASV_Z | (monocistronic vector) |
| Lane 2: LASV_NP | (monocistronic vector) |
| Lane 3: LASV_NP(3′ His-tag) | (monocistronic vector) |
| Lane 4: LASV_Z + NP | (bicistronic vector) |
| Lane 5: LASV_Z + NP(3′ His-tag) | (bicistronic vector) |
| Lane 6: LASV_Z + GPC | (bicistronic vector) |
| Lane 7: LASV_Z + GPC(Flag) | (bicistronic vector) |
| Lane 8: LASV_Z + NP(3′ His-tag) + GPC | (tricistronic vector) |
| Lane 9: LASV_Z + GPC(Flag) + NP(3′ His-tag) | (tricistronic vector) |

The LASV Z protein is minimally required to produce VLPs.

Protein content assay in VLP preparations (per four 15-cm cell culture dishes at 1.7×10⁷ cells/dish):

LASV Z VLP: 6.469 mg total protein (exemplified in lane 1 of FIG. 9). LASV Z+GPC VLP: 7.211 mg total protein.

### Example 4

Development of a regulatory compliant VLP-based vaccine to Lassa hemorrhagic fever.

Prior LASV vaccine strategies have employed gamma-irradiated LASV, attenuated reassortant arenaviruses, and recombinant vaccinia, vesicular stomatitis, yellow fever, and Venezuelan equine encephalitis virus-like replicon particles expressing LASV antigens. Although partial or complete protection was achieved with some vaccine candidates in guinea pig and non-human primate (NHP) models, all approaches tested lacked the safety and regulatory compliance necessary to generate a safe, well tolerated, broadly protective, mass produced, and cost effective vaccine against LASV. Most of these issues can be addressed by the development of a mammalian cell-derived VLP-based Lassa vaccine.

To this end, the instant inventors have designed a mammalian expression vector system with features allowing for the enhanced production of large quantities of VLP in transfected cells (described in Examples). The major immunological determinants of LASV are its glycoprotein complex, arising from the proteolytic cleavage of GPC into GP1, GP2 and the associated signal peptide (SSP), as well as the nucleoprotein (NP). Formation and release of LASV virions requires expression of the viral Z matrix protein; this protein alone is sufficient to generate VLPs, which can be seen as empty particles budding from cells in electron micrographs. Among other co-expression analyses, co-expression of Z, GPC, and NP genes in the same cell according to the instant invention resulted in the production and release of VLPs containing all three of these proteins. Lassa VLPs comprised of Z and GPC were also generated.

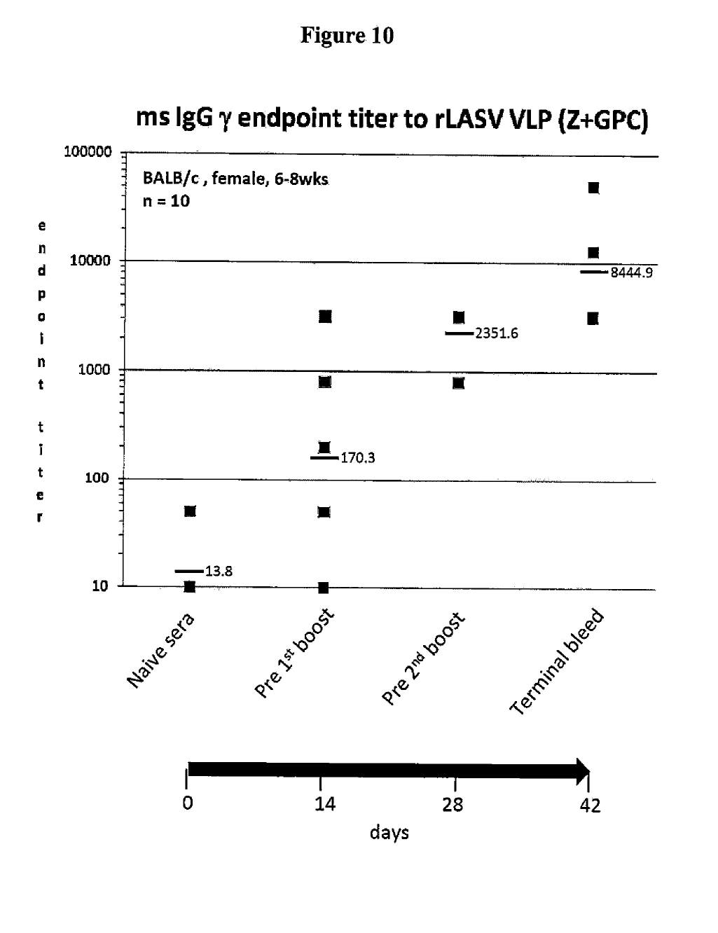

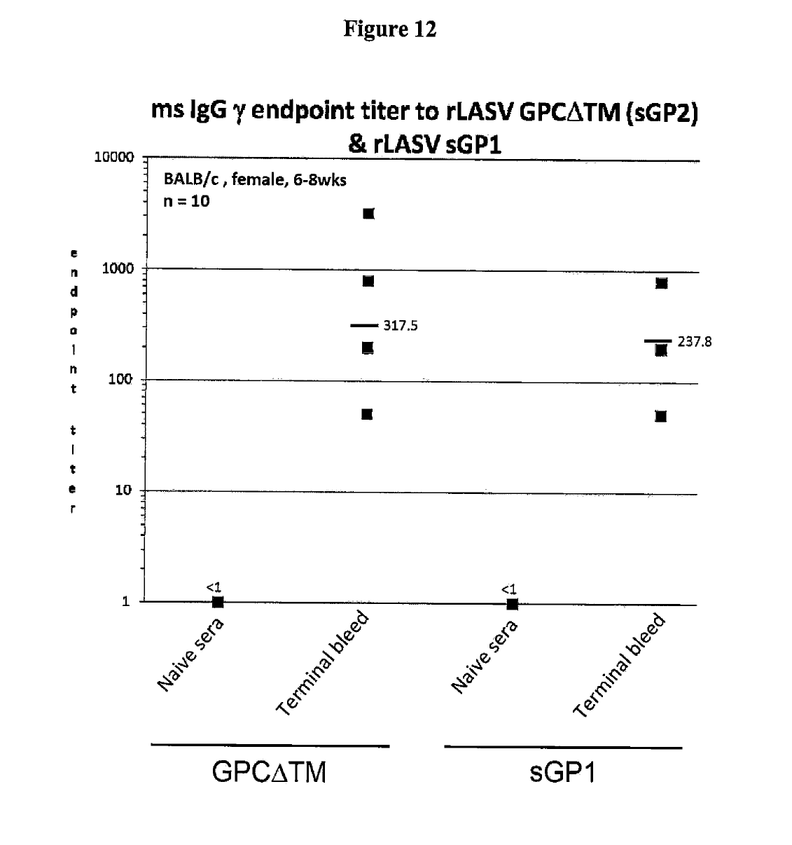

The resulting VLPs were biochemically characterized for total protein content, ratios of Z/GPC/NP, presence of host cell ribosomes and rRNA, and stability in a Tris-NaCl-EDTA (TNE buffer) formulation at 4° C. over 4 months. To date, BALB/c mice (mean, n=10) immunized with LASV VLP comprised of Z and GPC, using a prime+boost schedule (2 boosts, 2 weeks apart) with 10 μg of total pseudoparticle protein per mouse, without adjuvants, generated virion-specific endpoint IgG titers of 8400 (FIG. 10, terminal bleed), ˜240 (GP1-specific IgG, FIGS. 12) and ˜320 (GP2-specific IgG, FIG. 12—“GPCΔTM”) as determined by ELISA.

The specific parameters for the ELISA analyses for which data are shown in FIGS. 10-12 were as follows:

FIG. 10:
10 μg Lassa VLP (Z+GPC) in 100 mL total volume of TNE buffer was administered to each mouse intraperitoneally for vaccination/booster.
IgG (γ) titers assayed with a goat α-mouse IgG (γ)-HRP reagent.
ELISA plates coated with 100 mL/well of a 2 μg/mL Lassa VLP (Z+GPC) solution in PBS at 4° C. overnight.
Plates blocked with 200 μL/well 1× PBS/5% NFDM (non-fat dried milk)/0.02% Tween®-20 for 1 hour at room temperature.
Sera serially diluted 4-fold (1:50 and up) in 1× PBS/5% NFDM/0.02% Tween®-20/1% FBSΔ.
100 μL diluted sera incubated in blocked plates 1 hour at room temperature.
Plates incubated with goat α-mouse IgG (γ)-HRP reagent (KPL, Gaithersburg, Md.) at 1:2,500 dilution (0.4 μg/mL), 1 hour at room temperature in 1× PBS/5% NFDM/0.02% Tween®-20/1% FBSΔ.
TMB (tetramethylbenzidine) ELISA substrate applied and reacted for 5 minutes at 27° C., then stopped with 0.5 N H₂SO₄.
Plates read at 450 nm.

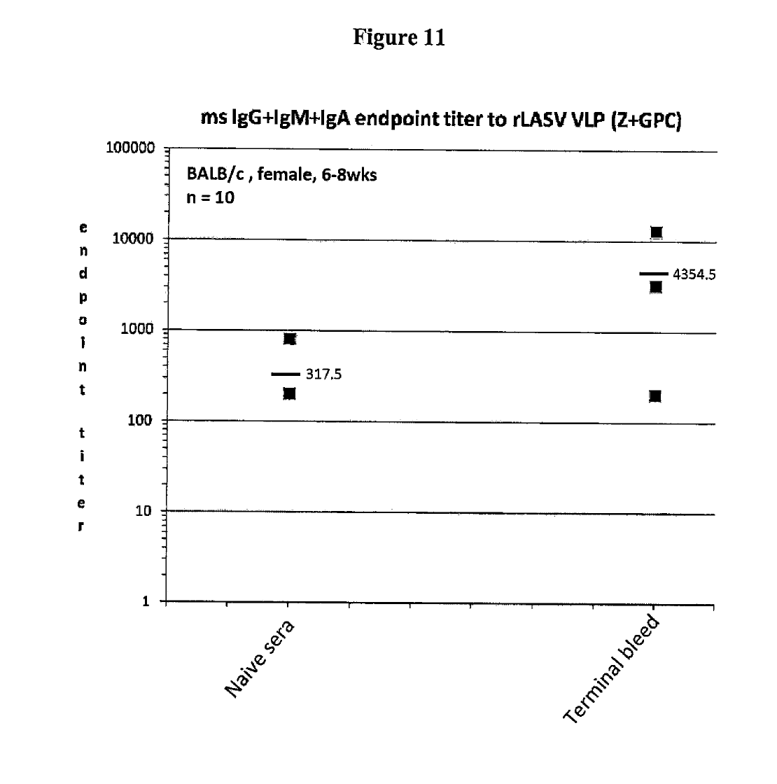

FIG. 11:
10 μg Lassa VLP (Z+GPC) in 100 mL total volume of TNE buffer was administered to each mouse intraperitoneally for vaccination.
IgG+IgM+IgA titers assayed with a goat α-mouse IgG+IgM+IgA-HRP reagent.
ELISA plates coated with 100 mL/well of a 2 μg/mL Lassa VLP (Z+GPC) solution in PBS at 4° C. overnight.
Plates blocked with 200 μL/ well 1× PBS/5% NFDM/0.02% Tween®-20 for 1 hour at room temperature.
Sera serially diluted 4-fold (1:50 and up) in 1× PBS/5% NFDM/0.02% Tween®-20/1% FBSΔ.
100 μL diluted sera incubated in blocked plates 1 hour at room temperature.
Plates incubated with goat α-mouse IgG+IgM+IgA-HRP reagent (KPL) at 1:2,500 dilution (0.4 μg/mL), 1 hour at room temperature in 1× PBS/5% NFDM/0.02% Tween®-20/1% FBSΔ.
TMB ELISA substrate applied and reacted for 5 minutes at 27° C., then stopped with 0.5 N H₂SO₄.
Plates read at 450 nm.

FIG. 12:
10 μg Lassa VLP (Z+GPC) in 100 mL total volume of THE buffer was administered to each mouse intraperitoneally for vaccination.
IgG (γ) titers assayed with a goat α-mouse IgG (γ)-HRP reagent.
ELISA plates coated with 100 mL/well of a 2 μg/mL Lassa sGP1 or sGP2 solution in PBS at 4° C. overnight.
Plates blocked with 200 μL/well 1× PBS/5% NFDM/0.02% Tween®-20 for 1 hour at room temperature.
Sera serially diluted 4-fold (1:50 and up) in 1× PBS/5% NFDM/0.02% Tween®-20/1% FBSΔ.
100 μL diluted sera incubated in blocked plates 1 hour at room temperature.
Plates incubated with goat α-mouse IgG (γ)-HRP reagent (KPL) at 1:2,500 dilution (0.4 μg/mL), 1 hour at room temperature in 1X PBS/5% NFDM/0.02% Tween®-20% FBSΔ.
TMB ELISA substrate applied and reacted for 5 minutes at 27° C., then stopped with 0.5 N H₂SO₄.
Plates read at 450 nm.

Endpoint titers were measured from those wells that received the highest serum dilution while also showing an absorbance₄₅₀ greater than three standard deviations from the mean absorbance₄₅₀ as measured from a series of control wells.

Initial results indicate that a Lassa VLP-based vaccine candidate is immunogenic, safe, and well tolerated in a murine model. The immunogenicity in BALB/c mice of Lassa VLPs comprised of GPC, NP, and Z proteins is currently being characterized. Additional studies will focus on establishing parameters that elicit a broad protective response against lethal Lassa challenge in Lassa VLP vaccinated animal models.

### Example 5

LASV Gene Expression and Incorporation in VLP

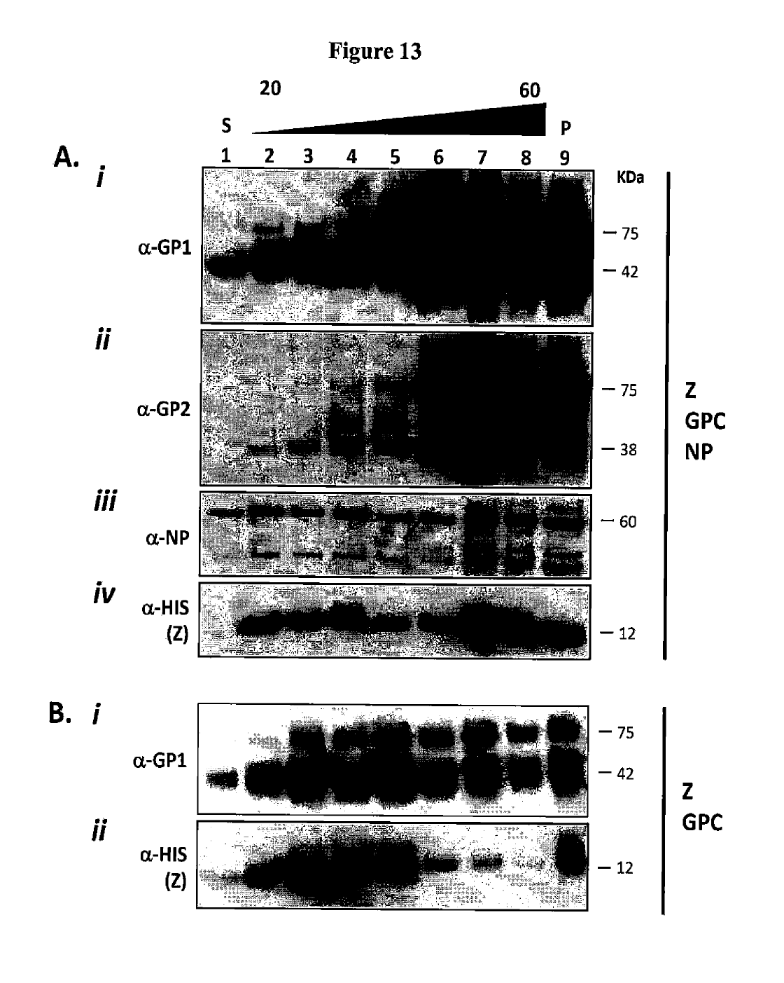

Transient transfection of HEK-293T/17 cells with LASV GPC, NP, and Z gene constructs resulted in high level expression of all proteins, including their known post-translational processing. The glycoprotein complex (GPC) was detected as a 75 kDa polyprotein precursor in transfected cell extracts, and in VLP preparations (FIG. 13Ai, Aii, Bi lanes 2-9). Similarly, the proteolytically processed GP1 and GP2 subunits were detected in cell extracts and in purified VLP (FIG. 13Ai, Aii, Bi lanes 2-9) as 42 and 38 kDa glycosylated species, respectively. In VLP cell culture supernatants cleared by ultracentrifugation, the soluble LASV GP1 isoform was also detected at high levels (data not shown). Nucleoprotein (NP) was mainly detected as a 60 kDa species, with smaller fragments identified, namely a 24 kDa protein corresponding to a proteolysis product generated during LASV infection in vitro (FIG. 13Aiii lanes 2-9). The nucleoprotein was largely absent from the extracellular milieu unless the Z matrix protein was co-expressed (FIG. 13Aiii, Aiv, lanes 2-9). A minor NP band could be detected in sucrose gradient fractions lacking VLP, as assessed by lack of GP2 and Z matrix protein (FIG. 13Aiii, lane 1). The Z matrix protein was detected in cell extracts and in VLP preparations, as a 12 kDa protein (FIG. 13Aiv, Bii, lanes 2-9). An N-terminal 6X-HIS tagged Z protein gene variant starting at amino acid position +3 that disrupted the known myristoylation domain also expressed at high levels, but failed to generate VLPs, as determined by lack of detection of the protein in cell culture supernatants. To determine if tagged arenaviral gene sequences benefited overall expression levels and incorporation into VLP, a series of matrix experiments were performed that combined native and/or 6X-HIS or FLAG epitope tags. Only the addition of a 6X-HIS tag to the C-terminus of the Z gene did not affect its expression and incorporation into VLP. Addition of C-terminal tags to GPC or NP resulted in lower expression levels and resulting incorporation into VLP. In some cases these tags led to unexpected and untoward proteolytic processing.

Large Scale Generation of LASV VLP

Generation of LASV VLP was scalable from 6-well plates through 15-cm cell culture dishes, with linear volumetric increases in particle yields (data not shown). Production of VLP for biochemical characterization and in vivo studies was performed in multiple 15-cm culture dishes, which routinely yielded an average of 2 mg of total VLP protein per dish, as determined by Micro BCA™ (Pierce) and SDS-PAGE. VLP generated from expression of LASV Z, GPC, and NP gene constructs resulted in particles with higher densities than those produced by expression of Z and GPC alone, as assessed by relative levels of each viral protein throughout the sucrose density spectrum (FIG. 13A, B, lanes 2-9). The majority of Z+GPC+NP VLP sedimented between 30 and 60% sucrose (FIG. 13Ai-iv, lanes 4-8), whereas Z+GPC VLP were present in ˜25-40% sucrose fractions (FIG. 13Bi, ii, lanes 3-5). Surprisingly, Z+GPC VLP sedimenting through 30-60% sucrose contained progressively lower levels of Z matrix protein (FIG. 13Bii, lanes 6-8) than counterparts containing both NP (FIG. 13Aiv, lanes 6-8) and Z. In both types of VLP preparations, a considerable insoluble fraction pelleted through 60% sucrose and could only be dissolved in reducing SDS-PAGE buffer (FIG. 13Ai-iv, Bi-ii, lane 9).

Effects of ASV Gene Expression on Mammalian Cell Morphology and Viability

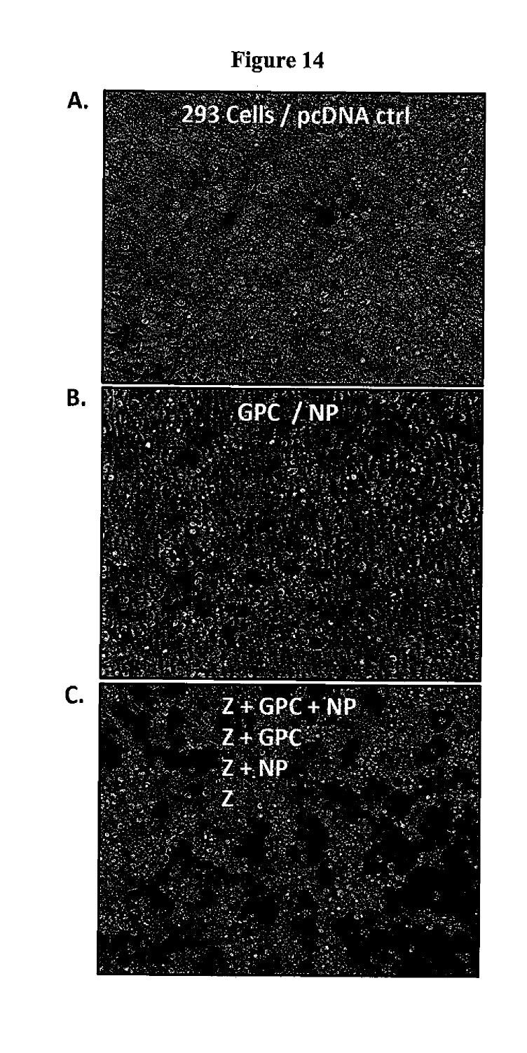

Expression of LASV GPC or NP alone did not induce significant morphological changes in 293T/17 cells through 72 hours post-transfection when compared to untransfected, mock transfected, or vector-only transfected cells, as assessed by light microscopy (FIGS. 14A, B). By contrast, inclusion of Z matrix gene protein in transfection experiments resulted in significant morphological changes, marked by elongation of cells by 24 hours, with significant detachment from the poly-D-lysine coated culture surface by 48 hours, resulting in large areas of monolayer breakdown (FIG. 14C). Cellular cytotoxicity was measured by MTT assays, and chromosomal DNA fragmentation analysis was employed to determine gross apoptotic or necrotic cell death mechanisms. Triplicate MTT experiments verified that single LASV NP, GPC, and GPC-FLAG gene expression did not result in significant cellular cytotoxicity when compared to vector transfected and untransfected 293T/17 cell controls. The inclusion of LASV Z or Z3′HIS in transfections experiments, alone or in combination with any other LASV gene construct resulted in significant levels of cytotoxicity, as measured by reduced OD₅₆₂ levels in MTT assays, with p<0.05 to p<0.001. Despite significant differences in MTT assays among transfected LASV gene combinations, TAE-agarose gel analysis showed lack of visible DNA fragmentation after a 72-hour transfection.

LASV VLP Contain a Multitude of Cellular Proteins in Addition to Viral Polypeptides

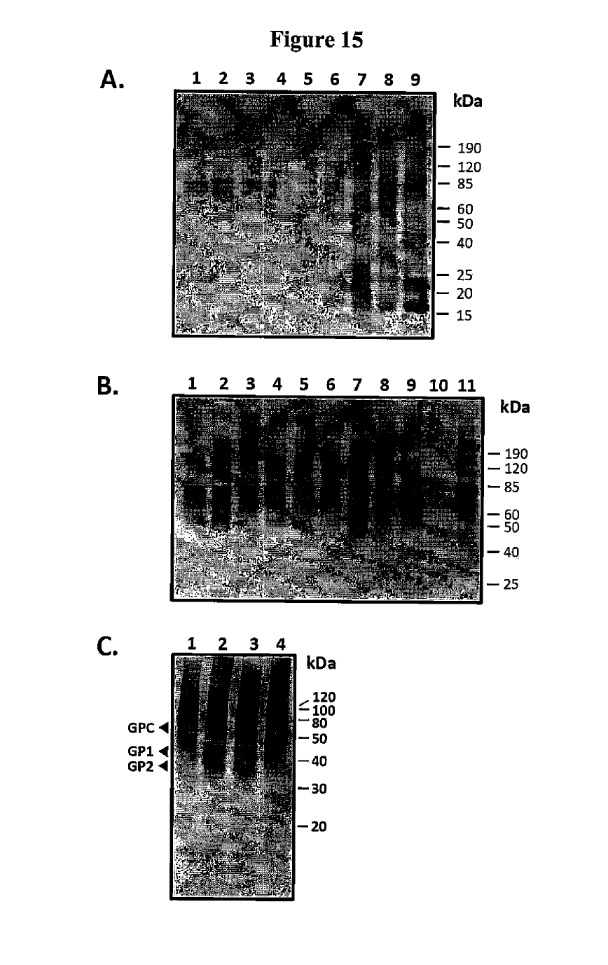

Analysis of sucrose gradient-purified LASV VLP by SDS-PAGE and Coomassie BB-R250 staining revealed a multitude of proteins, in addition to the expected viral polypeptides at ˜40 kDa (GP1 and GP2), 60 kDa (NP), and 12 kDa (Z) (FIG. 15A, lanes 1-9). These additional proteins are host cell-derived polypeptides, and range from ˜20 kDa to 200 kDa in size. Supernatants of mock- or pcDNA3.1+:intA-transfected cells did not yield detectable levels of PEG-6000/NaCl and sucrose cushion and/or gradient centrifugation-derived proteins, as determined by Micro BCA™ and SDS-PAGE analyses (data not shown). Glycan analysis using a wide range of lectins revealed that a significant number of non-viral proteins incorporated into LASV VLP are glycoproteins (FIG. 15B, lanes 1-9). Lectin binding specificity was assessed by lack of binding to LASV NP, GP1, and GP2 proteins generated in E. coli (FIG. 15B, lane 10). Lectin binding to glycosylated proteins included in the DIG Glycan Differentiation Kit (Roche) was included as a positive control (FIG. 15B, lane 11). A similar lectin binding analysis was obtained with VLP purified through 20% sucrose cushions containing Z alone, Z+GPC+NP, Z+GPC, or Z+NP (FIG. 15C, lanes 1-4), with the exception that additional diffuse bands could be discerned in VLP containing LASV glycoproteins (FIG. 15C, lanes 2-3).

LASV VLP glycoproteins display heterogeneous glycosylation

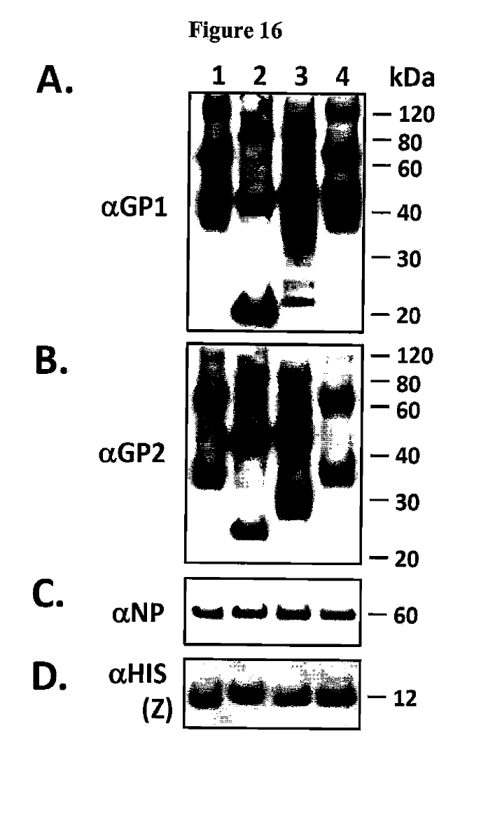

LASV VLP containing Z+GPC+NP were treated with N-Glycosidase F (PNGase-F), Endoglycosidase H (Endo-H), or Neuraminidase to assess gross glycosylation patterns. Experiments were performed with non-denatured (FIG. 16) and with heat-denatured VLP (data not shown), with identical results. PNGase-F completely removed glycans from GP1 and GP2, as well as from unprocessed GPC, as determined by mobility shifts from 42 to 20 kDa for GP1, 38 to 22 kDa for GP2, and from 75 to 42 kDa for GPC (FIG. 16A, B, lane 2). By contrast, Endo-H removed glycans from GP1, but to a much lesser extent than from GP2. Multiple bands were detected with anti-GP1 mAb in Endo-H treated LASV VLP containing GPC, ranging between 22 and 42 kDa, whereas probing of the same reactions with anti-GP2 mAbs revealed a relatively heterogeneous GP2 species at approximately 30 kDa (FIG. 16A, B, lane 3). Treatment of LASV VLP with Neuraminidase resulted in GP1 and GP2 glycosylation patterns similar to those obtained with untreated VLP (FIG. 16A, B, lane 4 versus lane 1). Treatment of LASV VLP with all three deglycosydases did not affect the mobility of NP (FIG. 16C, lanes 1-4) and Z proteins (FIG. 16D, lanes 1-4). In addition to deglycosylation of monomeric glycoproteins and unprocessed GPC, mobility shifts were readily detected for the approximately 120 kDa species likely composed of previously characterized trimerized glycoprotein monomers resistant to denaturation with SDS, reducing agents, and heat (FIG. 16A, B, lanes 3-4).

LASV VLP do not package cellular ribosomes

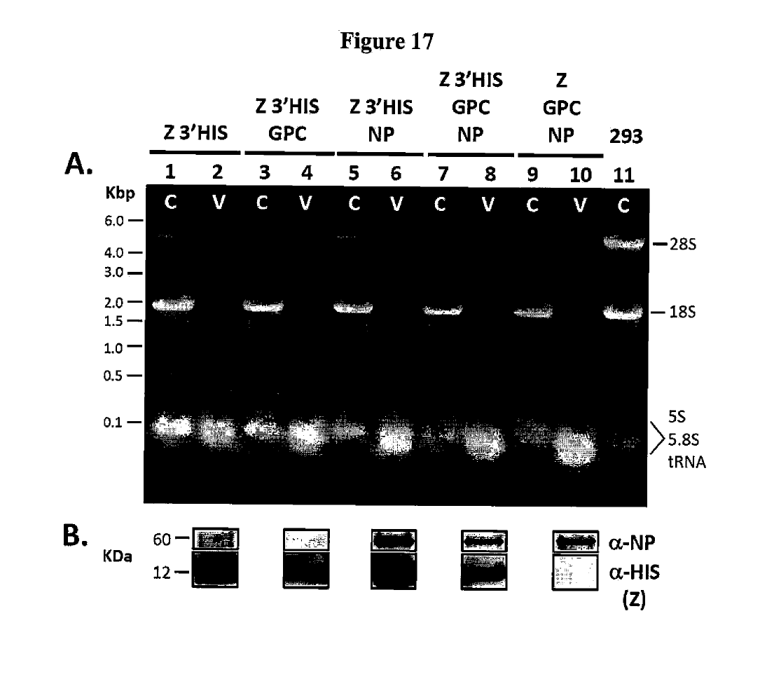

Ribonucleic acid content in LASV VLP generated in HEK-293T/17 cells lacked 18S and 28S ribosomal RNA (rRNA) species, as assessed by denaturing agarose gel electrophoresis, irrespective of the LASV gene combination (FIG. 17A, lanes 2, 4, 6, 8, 10). A low molecular weight RNA species of approximately 75 base pairs or less corresponding in size range to cellular tRNAs could be readily detected in VLP preparations containing either Z alone, or in combination with NP and GPC (FIG. 17A, lanes 2, 4, 6, 8, 10). This species was not detected in mock- or pcDNA3.1+:intA-transfected cell supernatants extracted with Trizol® reagent (data not shown). The 28S and 18S ribosomal RNA bands were present in total cellular fractions obtained from cells transfected with varying LASV gene constructs, although the 28S/18S ratio was significantly reduced when compared to the pcDNA3.1+:intA-transfected cell control (FIG. 17, lanes 1, 3, 5, 7, 9, versus lane 11). To verify that input LASV VLP used in RNA analysis contained the respective viral proteins, an aliquot of purified pseudoparticles were subjected to western blots analysis with anti-NP, anti-HIS (Z), and anti-GP2 antibodies. Western blot analysis revealed that input LASV VLP expressed the respective proteins of interest (FIG. 17B, lanes 2, 4, 6, 8, 10).

LASV VLP are morphologically similar to native virions

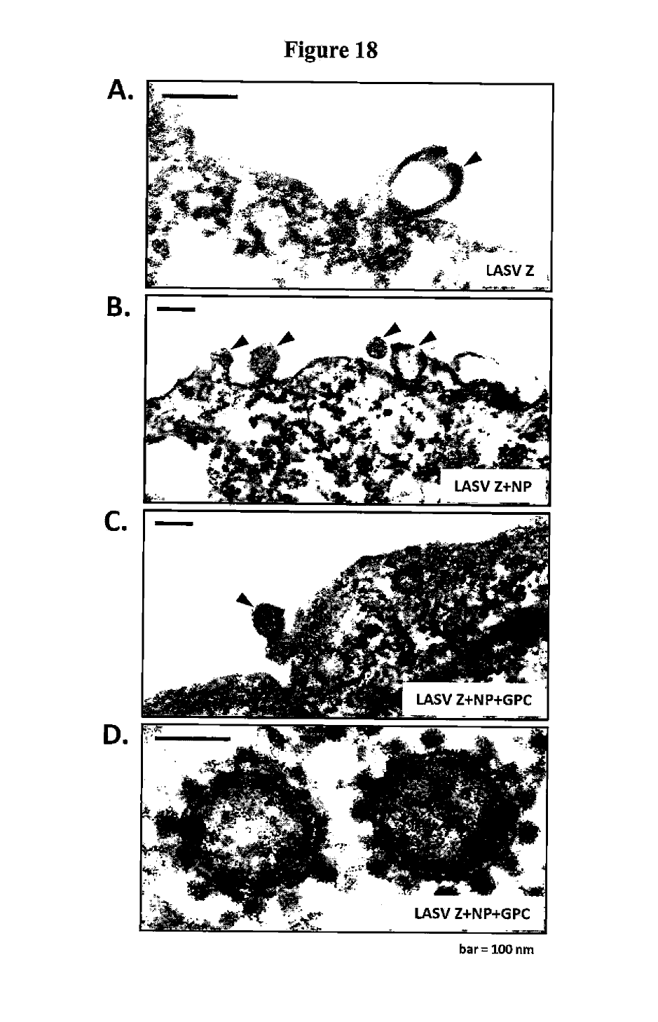

Electron microscopy (EM) was employed to dissect the morphological properties of VLP generated by expression of Z matrix protein alone, or in combination with NP and GPC. Expression of LASV Z gene alone was sufficient to induce budding of low electron density empty VLP from the surface of transfected cells (FIG. 18A). By contrast, expression of Z in conjunction with NP or NP+GPC resulted in the generation of electron dense VLP with granular material associated with the pseudoparticles (FIG. 18B-D). The granular structures were similar to cellular ribosomes in size (FIG. 18D), but identification of these subcellular organelles as the granular elements, as well as their physical association and incorporation in VLP were not determined in these studies. LASV VLP displayed pleiomorphic morphology by EM, with sizes ranging from 100-250 nm, and enveloped by bilayer structure (FIG. 18D).

LASV VLP display glycoprotein resistance to proteolysis by trypsin

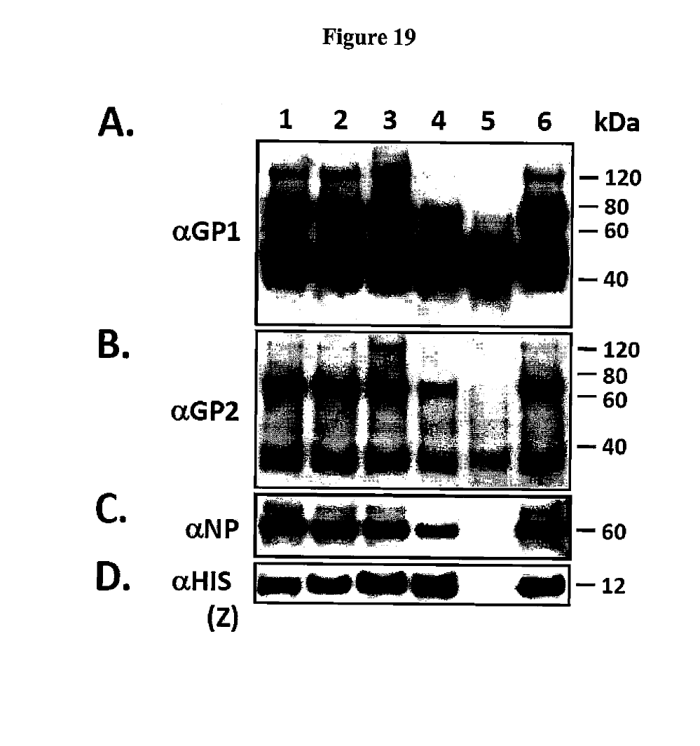

Trypsin protection assays were employed to characterize protein content and structural compartmentalization of LASV antigens. Treatment of VLP with soybean trypsin inhibitor alone, with 1% Triton® X-100 alone, or with soybean trypsin inhibitor and trypsin had no effect on the integrity of GP1, GP2, Z, and NP proteins when compared to untreated controls (FIGS. 19A-D, lanes 2, 3, 6 versus lane 1). Treatment of VLP with trypsin alone completely digested the approximately 120 kDa trimerized GP1 species and partially digested unprocessed GPC, while monomeric GP1 remained largely resistant to the protease (FIG. 19A, lane 4). Similarly, trypsin completely digested the approximately 120 kDa trimerized GP2 species, but only partially digested monomeric GP2 (FIG. 19B, lane 4). Trypsin treatment of intact LASV VLP did not significantly affect detection of NP and Z proteins (FIGS. 19C-D, lane 4). Whereas, treatment of LASV VLP with Triton® X-100 and trypsin resulted in increased digestion of both glycoproteins, but significant levels of GP 1 and GP2 could still be detected (FIG. 19A-B, lane 5). Under these conditions, both NP and Z proteins were completely digested by trypsin (FIG. 19C-D, lane 5). Digestion of intact VLP in the presence of soybean trypsin inhibitor completely prevented digestion of any form of the exposed glycoprotein complex (FIG. 19A-B, lane 6).

LASV VLP are immunogenic in mice and induce a mature IgG response after prime plus two boosts intra peritoneal immunizations

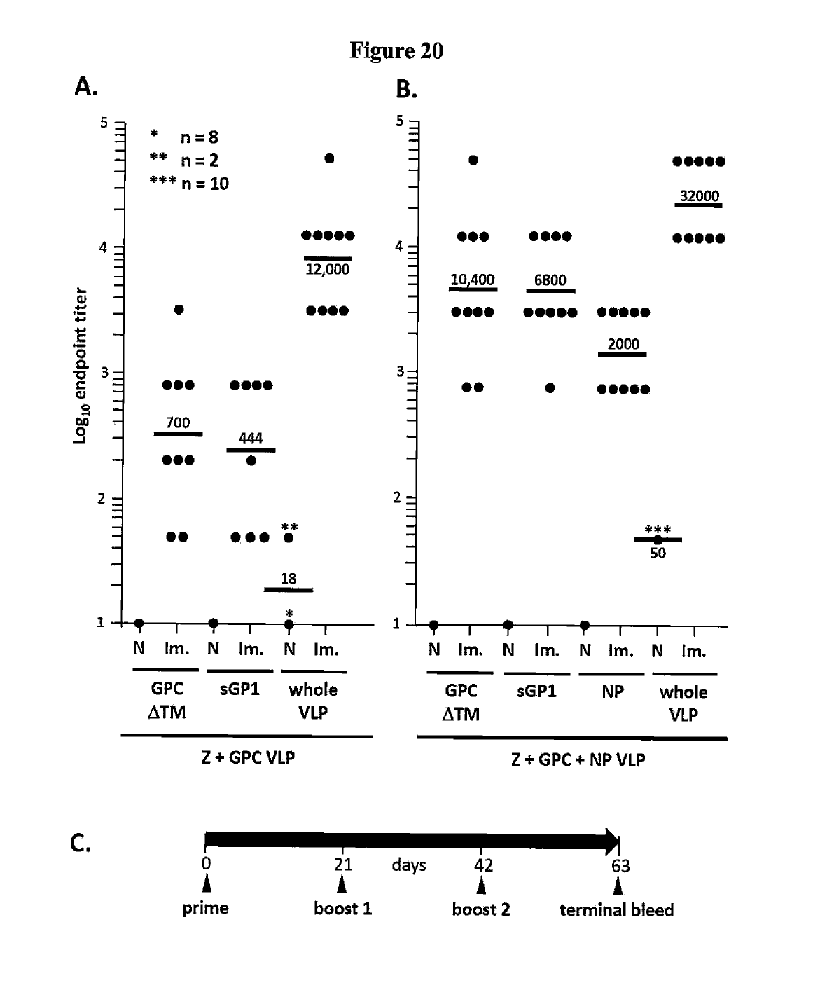

Mice were immunized with LASV VLP containing Z and the glycoprotein complex (Z+GPC), or including the NP protein (Z+GPC+NP), in the absence of an adjuvant using a prime+2 boosts schedule, 3 weeks apart. Total LASV antigen-specific IgG levels were assessed by ELISA on VLP, NP, GP1, or GP2 coated plates. Three weeks following a single 10-μg dose administration of VLP, a significant number of mice had generated IgG-specific responses to LASV antigens (data not shown). Following a homologous first boost all animals generated more robust LASV protein-specific IgG, which was further enhanced in all animals after a second boost, and assessed terminally 63 days post first immunization (FIG. 20). The IgG response against both types of whole VLP was significantly more robust than to individual antigens, with mean endpoint titers of 12,000 and 32,000 for Z+GPC and Z+GPC+NP VLP, respectively. Most notably terminal IgG titers against GP 1 and GP2 in Z+GPC+NP VLP were approximately 15 fold higher than to Z+GPC VLP. Most animals immunized with Z+GPC VLP responded poorly to both glycoproteins, with 2/10 and 3/10 producing endpoint titers of 50 to GP2 and GP1, respectively, with only one animal registering an IgG titer of 3200 to GP2. Animals immunized with Z+GPC+NP responded well to both glycoproteins, with mean titers of 10,400 and 6,800 for GP2 and GP1, respectively, with 4/10 animals registering greater than 12,800 endpoint titer to each glycoprotein. Titers to Z matrix protein were not determined in these studies.

LASV patient sera specifically recognize VLP antigens in conformational and individual recombinant viral proteins

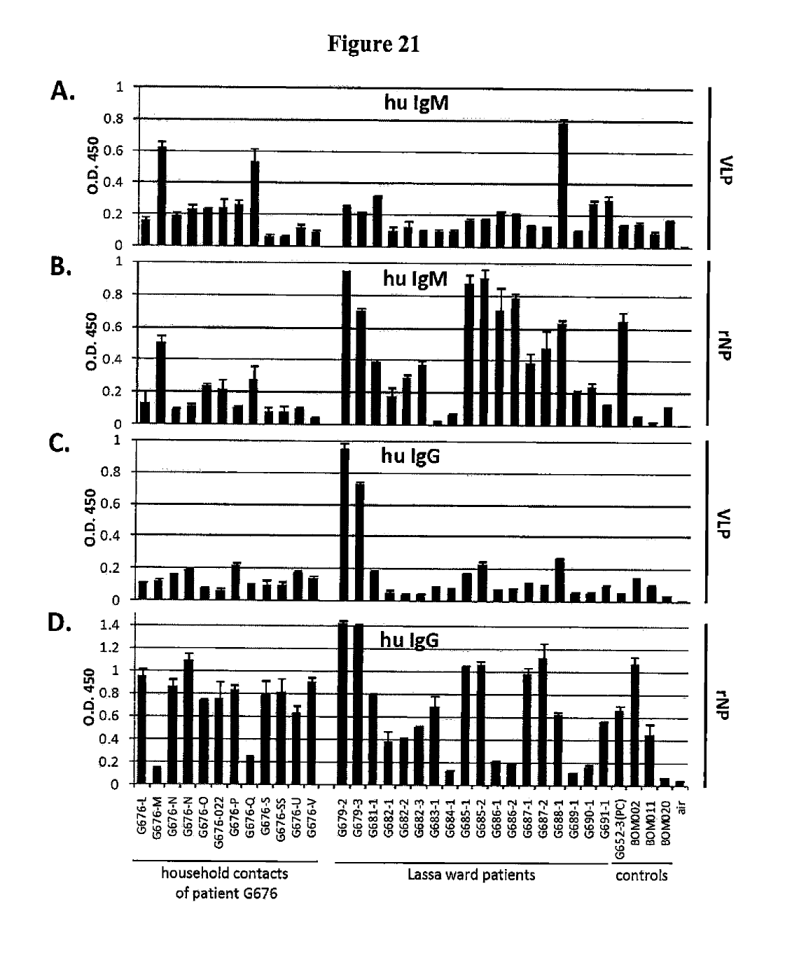

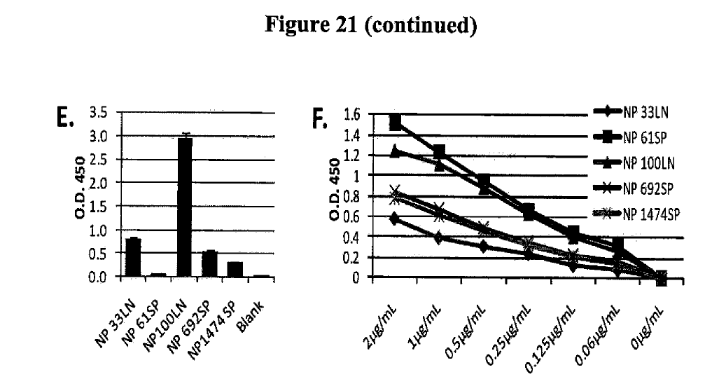

LASV-specific IgM and IgG titers in convalescent subjects and patient sera were used to characterize humoral responses to quasi-native viral epitopes on VLP. A subset of sera reacted with LASV VLP in either IgM or IgG detection platforms, but usually not both (FIGS. 21A, B). None of the presumed negative control samples showed reactivity to LASV VLP in these assays (FIG. 21A, B, BOM002, BOM011, BOM020). The positive control serum did not react with LASV VLP in the present format (FIGS. 21A, C, G652-3(PC)), although it bound to rNP (recombinant NP) in both IgM and IgG assays format (FIGS. 21B, D, G652-3(PC). Overall, there was poor correlation between LASV VLP and rNP detection of viral protein-specific IgG and IgM in human sera. Characterization of LASV NP epitope presentation in the context of a VLP was performed by ELISA using a series of mAbs raised against recombinantly expressed LASV NP. All five NP-specific mAbs showed differential binding levels to NP in VLP (FIG. 21E), despite all capturing recombinantly expressed NP in solution at the concentration tested (FIG. 21F).

Discussion

Lassa virus-like particles were generated to contain the major immunological determinants of the virus, resembled native virions structurally, and were immunogenic in mice. Plasmid vectors well suited for high level expression of recombinant proteins in mammalian cells through combination of rational design and proven genetic elements have resulted in superior yields of LASV VLP. These vectors afford the possibility of developing a VLP-based vaccine candidate in mammalian cell systems at low cost per dose, using transient expression technologies. Despite incorporation of all LASV proteins into VLP, both glycoproteins were present at significantly higher levels in most sucrose density fractions than either NP or Z (FIG. 13). Incorporation of high levels of both glycoproteins in VLP may be beneficial in a vaccine platform, as these viral components alone have been shown to confer full protection against challenge with lethal doses of live LASV in non-human primates. Despite the high levels of glycoprotein incorporation into LASV VLP, addition of the nucleoprotein may be of critical importance in establishing more robust and long lived immunity against Lassa virus. Previous studies have demonstrated physical interaction between the glycoprotein complex, the Z matrix, and nucleoproteins during viral biogenesis. Thus, these natural interactions are greatly beneficial since they result in the generation of VLP that package all viral immunogenic and protective determinants from a single set of transiently transfected recombinant LASV genes. In these studies we employed the human endothelial kidney cell line HEK-293T/17 for its high levels of transfectability, expression of recombinant proteins from human cytomegalovirus (hCMV) promoter-driven gene constructs, and resulting yields of LASV VLP. During the course of this work we have also established the value of using HEK-293T/17 as an indicator cell line. The profound morphological changes manifested by the cell line upon expression of LASV Z matrix protein is a good indicator of transfection efficiency and overall production levels of resulting VLP (FIG. 14). Despite significant adverse metabolic effects on cells expressing LASV proteins and generating budding VLP, culture viability remains high (mean=70%) at the time of harvest. This desirable aspect of mammalian cell culture-based production is beneficial in downstream purification processes, by reducing host cell components that must be eliminated from the final purified product, namely cellular proteins, DNA, RNA, and lipids. Other expression platforms cannot be easily employed in the generation of LASV VLP where the glycoprotein complex precursor is used to incorporate processed GP1 and GP2. Truncated versions of the GPC precursor lacking the transmembrane domain have been generated in E. coli (unpublished data from the Viral Hemorrhagic Fever Research Consortium) and in baculovirus expression systems. In E. coli, the protein is neither glycosylated nor cleaved into GP1 and GP2 subunits. In insect cells the protein is glycosylated but is not cleaved. Both expression systems lack the critical SKI-1/S1P subtilase responsible for co-translational processing of the LASV GPC precursor in mammalian cells. Despite the possibility of co-expressing the subtilase in heterologous systems to facilitate processing of GPC precursor, the glycosylation profile of GP1 and GP2 subunits may play a critical role in the structure and function of each protein in vivo. Thus, a mammalian expression system remains a highly attractive platform for the development of an arenaviral VLP-based vaccine.

We have determined in these studies that LASV VLP contain, in addition to the intended viral polypeptides, a plethora of host cell membrane proteins, presumably acquired during budding from the cell membrane or other intracellular lipid bilayer-containing structures, such as the Golgi apparatus. A significant portion of the viral envelope protein content is made up of host cell glycoproteins, as determined by a broad glycan binding analysis performed on sucrose sedimented fractions. The host cell glycoprotein composition varies along the gradient spectrum, with one particular ˜48 kDa protein highly represented in the 20% fraction, but much less evident in the 30% and denser fractions (FIG. 15A). This protein is also present at high levels in the input supernatant fraction, which is largely devoid of VLP, as determined by the absence of Z protein detection. This protein resolved as a single sharp band on SDS-PAGE and by glycan analysis, and falls outside the range of GP1, GP2, and unprocessed GPC. It has been reported by Schlie et al. (2010, J. Virol. 84:3178), and others, that transfection of mammalian cells with a full length LASV GPC construct is sufficient to generate GP VLP containing glycoprotein spikes. In our studies, and despite the presence of a monomeric GP1 species in the least dense sucrose fraction corresponding mainly to input precipitated VLP, GP2 could not be detected with long exposures of blots shown in FIG. 13. Thus, it is unlikely that the prominent glycoprotein species detected at 48 kDa could be an isoform of LASV GP VLP. A similar pattern of cellular glycoproteins incorporated into LASV VLP was detected in purified particles generated from expression of Z alone, or in combination with GPC and NP (FIG. 15C). In Z+GPC or Z+GPC+NP VLP, a diffuse lectin binding pattern could be detected between 38 and 42 kDa that was absent from VLP that did not express the glycoprotein complex. This pattern was detected in addition to the prominent cellular glycoprotein at ˜48 kDa in all VLP formats (FIG. 3C). The majority of detected cellular glycoproteins incorporated into LASV VLP ranged from 30 to greater than 220 kDa in mass. Recently, Moerdyk-Schauwecker et al. (2009, Virol. J. 6:166) characterized the spectrum of mammalian host cell proteins incorporated into vesicular stomatitis virus (VSV), an enveloped virus, during viral biogenesis. In total, 64 proteins of host cell origin were identified via a proteomics approach coupled with mass spectrometry (MS). Of the 64 host cell proteins identified in those studies, 10 were glycoproteins. Although a similar study has not been performed for any member of the arenaviridae, it is likely that some common host cell proteins are packaged among a wide array of viral classes, and some of these proteins may even play functional roles during viral infection and replication.

We had previously characterized the gross glycosylation profile of LASV GP1 in the context of a soluble isoform (sGP1) of this viral protein. In the present studies, we characterized LASV VLP-associated GP1 and GP2 glycosylation patterns. Glycoprotein 1 associated with VLP generated essentially the same glycosylation pattern as sGP1, with only partial deglycosylation by Endo H, and insignificant processing by Neuraminidase (FIG. 16A). These results point to a heterogeneous array of glycans on the surface of GP1 that include some high mannose and branched oligosaccharides. Glycoprotein 2 displayed a more heterogeneous glycan array with a highly homogeneous high mannose and hybrid oligosaccharide content that accounted for approximately 8 kDa of the fully processed mass of the protein, based on the detection of a relatively sharp 30 kDa species upon treatment with Endo H (FIG. 16B, lane 3). The remaining 7 kDa of glycan content could be removed by treatment of the protein with PNGase F, but not with Neuraminidase (FIG. 16B, lanes 3-4). The micro- and macroheterogeneity in both GP1 and GP2 N-linked glycosylation has not been characterized, but the highly heterogeneous and distinct oligosaccharide patterns on each glycoprotein may have a functional role during viral infection. We have established through these studies that GP1 incorporated into LASV VLP is highly resistant to proteolytic digestion by trypsin (FIG. 19A, lanes 4-5), despite 13 predicted trypsin recognition sites on the polypeptide backbone (ExPASy proteomics server tools, PeptideCutter). Similarly, GP2 is resistant to digestion with trypsin, albeit to a lesser extent than GP1, even after solubilization of the pseudoparticle envelope with Triton® X-100 (FIG. 19B, lanes 4-5). The PeptideCutter tool in ExPASy predicted 25 recognition sites with high confidence in the GP2 polypeptide backbone. The glycoprotein complex spike is the most readily accessible viral antigen to the innate immune system and to circulating serum proteases. Thus, it is of paramount importance to the virus that the critical components required for binding and fusion to permissive host cells be preserved. The specific glycosylation patterns on GP1 and GP2 may play a functional role in this process. Although glycosylation characterization studies have not been reported on glycoproteins from native LASV virions, it is likely that a similar pattern would emerge from that reported herein. In the studies by Schlie et al. (2010), Proteinase K protection assays performed on glycoprotein-expressing VLP also revealed partial resistance of the GP2 component against degradation by the protease, although solubilization with Triton® X-100 in conjunction with protease resulted in complete digestion of the protein.

To characterize the structural compartmentalization of viral proteins in LASV, we performed trypsin protection assays in the absence or presence of the anionic detergent Triton® X-100 (FIG. 19). In the absence of detergent, trypsin completely digested non-reducible GP1 trimer, partially degraded unprocessed GPC, but had no effect of monomeric GP1 (FIG. 19A, lane 4). A similar digestion pattern was obtained for GP2 (FIG. 19B, lane 4). Addition of detergent to the reaction enhanced digestion of unprocessed GPC and had a minor effect on sensitivity of GP 1 to the protease (FIG. 19A, lane 5). Dissolution of the envelope by detergent resulted in more pronounced degradation of GP2 by trypsin, although a significant portion of the monomer could be detected (FIG. 19B, lane 5). Only treatment of LASV VLP with Triton® X-100 resulted in proteolytic degradation of both Z matrix and NP proteins. These results strongly support the model of a LASV VLP containing glycoprotein spikes on the surface of a lipid envelope, with an internal matrix of Z protein containing the nucleoprotein component. We have shown that the viral proteins NP, Z, GP1 and GP2 can be co-expressed in VLP. Protein-protein associations appear to be an important aspect to the formation of VLP. Schlie et al. (2010) reported that a co-localization of NP, Z, and GP occurs near the nucleus. Similarly, Eichler et al. 2004, Virus Res. 100:249) demonstrated that NP and Z co-localize in the cell. They also demonstrated that NP could be precipitated using an antiserum against Z and vice versa. Furthermore, Schlie et al. (2010) determined that NP did not influence the interaction of GP and Z, nor could an interaction between NP and GP be detected in the absence of Z in co-localization and immunoprecipitation experiments. However, pull-down experiments performed by Schlie et al. (2010) demonstrated an association between Z and GP, and Z and NP. Strecker et al. 2006 (Virol. J. 3:93) reported that Z myristoylation is important for binding to lipid membranes. Flotation experiments using wild-type Z protein and a form of Z mutated at the myristoylation site showed that the mutant remains localized in the cytosol, whereas the wild-type associated with the membrane. Thus, the interactions between Z and the membrane, and with GP and NP, allow for VLP formation with relevant proteins.

Another structural component of native LASV virions are host cell ribosomes that are packaged during virus assembly, presumably for enhanced viral mRNA translation in the early stages of cellular infection. To determine whether LASV VLP containing any combination of Z matrix, GPC, and NP proteins mediated the ability to package cellular ribosomes, total RNA was isolated from pseudoparticles and analyzed by denaturing RNA gel electrophoresis (FIG. 17). RNA was also isolated from the corresponding transfected cells and analyzed alongside VLP RNA. All VLP formats analyzed in these studies did not contain significant levels of the 28S and 18S ribosomal RNA (rRNA) species known to be critical components of mammalian ribosomes (FIG. 17, lanes 2, 4, 6, 8, 10). In some analyses RNA was purified from 1 mg of total purified VLP, and the entire purified nucleic acid fraction was analyzed by gel electrophoresis, without distinct ribosomal RNA bands visible (data not shown). Despite the lack of rRNA detection in LASV VLP, all pseudoparticle formats analyzed in these studies contained significant levels of low molecular weight RNA species of ˜75-200 nt, that co-migrated with cellular 5S (120 nt) and 5.8S (160 nt) rRNA, and transfer RNAs (˜75-95 nt). It is reasonable to assume that in native VLP the incorporation of host cell ribosomes would result in the co-packaging of critical tRNAs for translation of viral mRNAs. Although in these studies the exact nature of the packaged RNA species was not characterized in detail, the results suggest that multiple RNA species of ribosomal origin are incorporated into VLP. To confirm that ribonucleoproteins were not incorporated into virions we performed western blot analysis on VLP proteins using antibodies raised against U1 snRNP 70, La/SSB, and Ro/SSA, but none could be detected in pseudoparticles (data not shown). These studies also point to a critical presence of viral RNA polymerase and genomic RNA segments during replication for subsequent incorporation of host cell ribosomes into nascent viral particles. Despite the lack of detectable rRNA in LASV VLP comprised of any combination of LASV proteins analyzed in these studies, pseudoparticles that contained GPC and/or NP in addition to Z matrix protein were morphologically similar to native virions (FIG. 18B-D). These VLP were electron-dense particles with punctuate inclusions and appeared to associate with highly electron-dense subcellular organelles in the cytoplasm, possibly ribosomes (FIGS. 18C, D). The size of mammalian ribosomes is approximately 20 nm, in line with the size of the particles associated with nascent LASV VLP imaged in these studies (FIG. 18D). These subcellular structures could not be detected in VLP budding from the surface of cells transfected with Z matrix protein alone (FIG. 18A), which appeared empty and containing only an envelope structure, and which has been reported by others (Urata et al., 2006, J. Virol. 8:4191).

For immunizations, LASV VLP comprised of Z+GPC or Z+GPC+NP were formulated in PBS and used to immunize BALB/c mice, in a prime+2 boosts schedule, 3 weeks apart, in the absence of an adjuvant, and administered by i.p. injection. After a single immunization some animals showed a low level IgG response to individual LASV antigens (data not shown), with increasing antibody titers with each subsequent boost. ELISA analysis of terminal IgG titers showed a clear difference in the response levels against GP1, GP2, and whole VLP between Z+GPC and Z+GPC+NP pseudoparticles (FIGS. 20A, B). VLP containing all three proteins induced a significantly higher response to the glycoprotein components compared to Z+GPC VLP, with a 15 fold increase in titer against both GP1 and GP2. Likewise, the titers against whole Z+GPC+NP VLP were nearly 3 fold higher than to Z+GPC pseudoparticles (FIG. 20A, B).

Lastly, we attempted to use LASV VLP as a diagnostic tool for the detection of viral protein-specific IgM and IgG in the serum of convalescent subjects, patients from the Lassa ward, contacts from patients who succumbed to Lassa fever, and individuals not known to have had the febrile illness at any given time in their lives. The LASV antigen binding profile of these sera was extensively characterized using highly sensitive and specific recombinant protein-based diagnostics under development by the Viral Hemorrhagic Fever Research Consortium. The overall poor level of correlation observed in human serum IgM (r =0.3297; r²=0.1087) and IgG (r=0.6284; r²=0.3949) binding profiles between LASV VLP and recombinant proteins in these studies was not surprising. Recombinant LASV proteins currently employed in diagnostic assays were generated in bacterial or mammalian cell systems, as outlined in Branco et al. (2009, Virology J. 6:147) and Illick et al. (2008, Virology J. 5:161). Individually produced, purified, and characterized proteins were used alone or in combination to coat high protein binding ELISA plates for determination of serum IgM and IgG binding profiles. Thus, it would be expected that protein-protein interactions known to play a role during viral biogenesis and in the formation of LASV VLP result in presentation of different epitopes and conformations than in counterparts generated as individual polypeptides. The known interactions between Z, GPC, and NP proteins in a VLP format likely mask the presentation of relevant epitopes to which a given individual may have generated IgM and IgG. As a result, native presentation of antigens in the context of a VLP, even in the presence of low levels of the membrane solubilizing detergent Tween®-20, will likely not result in disruption of protein interactions necessary for the detection of epitope-specific serum antibodies. This is supported by the fact that all five NP-specific mAbs used in this analysis detected and captured recombinantly expressed NP in solution (FIG. 21F), albeit at different levels. In combination, these results strongly suggest that LASV proteins, in the context of a VLP, display epitopes that possibly mimic native conformation and presentation. These observations further support the use of LASV VLP as a vaccine platform by supplying a quasi-native antigen, thus allowing the innate and adaptive immune systems to preferentially target epitopes relevant for immune protection against the virus.

Methods

Cells, plasmids, antibodies

HEK-293T/17 cells (ATCC CRL11268) were maintained in complete high glucose Dulbecco's Modified Eagle Medium (cDMEM) supplemented with non-essential amino acids (NEAA) and 10% heat-inactivated fetal bovine serum (AFBS).

Plasmid constructs expressing LASV GPC and the backbone vector pcDNA3.1+zeo:intA were described elsewhere (Illick et al., 2008). Optimized Z and NP genes for expression were amplified from LASV Josiah infected VERO cell RNA, as previously outlined (Illick et al., 2008). The LASV-specific GP1 mAb L52-74-7A and GP2 mAb L52-216-7, which were generated against purified gamma-irradiated LASV, were used for immunoassays. Monoclonal antibody to poly-histidine (6X-HIS) was purchased from Invitrogen, Inc. LASV NP-specific polyclonal sera were generated in goats by immunizing animals with 100 μg of E. coli-generated protein per injection, using a prime+3 boosts strategy, followed by terminal bleeds (Bethyl Laboratories, Inc.). The LASV NP-specific goat IgG fraction was subsequently purified by affinity column chromatography with agarose beads coupled to NP immobilized by AminoLink® chemistry (Thermo Fisher Scientific, Inc., Rockford, IL). Horseradish peroxidase (HRP)-conjugated secondary antibodies specific for goat and mouse IgG were purchased from KPL (Gaithersburg, Md.). The NP-specific hybridomas NP 33LN, NP 100LN, NP 61SP, NP 692SP, and NP 1474SP were generated by fusion of the SP2/0-Ag14 myeloma cell line with splenic and mesenteric lymph node lymphocytes from BALB/c mice immunized with E. coli-expressed NP. Monoclonal antibodies were produced in serum free medium (PFHM II, Invitrogen), purified via Protein-G chromatography, quantitated by A280, BCA, and SDS-PAGE.

Transient expression of LASV gene constructs

Recombinant LASV protein expression was analyzed in HEK-293T/17 cells transiently transfected with mammalian expression vector DNAs, which were prepared using the Endo-Free PureLink HiPure plasmid filter maxiprep kit (Invitrogen, Carlsbad, Calif.). The negative control vector pcDNA3.1(+):intA was included in all transfections. Protein concentration was determined for each sample by A280 with A260 subtraction, and verified using a Micro BCA™ Protein Assay Kit, as outlined by the manufacturer (Thermo Scientific).

Generation and purification of LASV VLP

LASV VLP were generated by transfecting HEK-293T/17 cells in 6-well plates (for small scale analysis) or in 15-cm plates (for purification of multi-milligram quantities of VLP) using Lipofectamine 2000 (Invitrogen). Cells were seeded on plates coated with 50 μg/mL Poly-D-Lysine hydrobromide, and were transfected at >90% confluence. Monolayers were transfected with equimolar amounts of vector DNAs, and when required reactions were normalized for DNA content with empty pcDNA3.1(+):intA. Cell supernatants were harvested 4 days post transfection and were clarified by centrifugation at 4000× g for 20 minutes at room temperature. Clarified supernatants were transferred to Beckman polyallomer ultratubes and gently mixed with polyethylene glycol-6000 (Sigma/Fluka) and sodium chloride to final concentrations of 5% and 0.25M, respectively. Reactions were incubated at +4° C. overnight, followed by centrifugation for one hour at 15,000× g, +4° C., in an SW28 rotor, to pellet the precipitated VLP. Pellets were gently resuspended in 20 mM Tris, pH7.4, 0.1M NaCl, 0.1 mM EDTA (TNE), or in 1× PBS, pH 7.4, overlaid on 20% sucrose cushions, and centrifuged for 2 hours at 55,000 rpm, +4° C., in an SW60Ti rotor. Pellets were resuspended in TNE or PBS and VLP were further purified on 20-60% discontinuous sucrose gradients, as described above for sucrose cushions. VLP were removed from visible bands throughout the gradient, combined, diluted in TNE or PBS, and centrifuged for one hour at 15,000× g, +4° C., in an SW28 rotor, to pellet the purified VLP and to remove sucrose. Pellets were resuspended in TNE or PBS and allowed to dissolve fully at 4° C. overnight. VLP used for immunizations were filtered through 0.45-gm syringe filters before being assayed for protein content by Micro BCA. VLP preparations were stored at 4° C. in TNE or PBS at concentrations ranging from 200-3000 μg/mL. VLP for immunizations were tested for endotoxin levels with a high sensitivity Limulus Amebocyte Lysate (LAL) test (Sigma-Aldrich).

Western blot and densitometry analyses

Expression of LASV GP1, GP2, NP, and Z-3′HIS in VLP were confirmed by Western blot analysis using anti-LASV mAbs L52-74-7A, L52-216-7, Goat PAb to E. coli generated nucleoprotein, and anti-6X-HIS mAb, respectively. Secondary antibodies were horseradish peroxidase (HRP)-conjugated goat anti-mouse IgG (H+L) or rabbit anti goat IgG (H+L). Five to ten μg of total VLP protein were denatured, reduced, and resolved on 10% NuPAGE® Bis-Tris gels, according to the manufacturer's specifications (Novex, San Diego, Calif.). Proteins were transferred to 0.45-gm nitrocellulose membranes, blocked, and probed in 1× PBS, pH 7.4, 5% non-fat dry milk, 1% heat inactivated fetal bovine serum, 0.05% Tween®-20, and 0.1% thymerosal. Membranes were then incubated in LumiGlo® chemiluminescent substrate (KPL) and exposed to Kodak BioMax® MS Film. Developed films were subjected to high resolution scanning for densitometry analysis. Quantification of band intensity was performed using National Institutes of Health ImageJ 1.41o software, and following the procedure outlined in www.lukemiller.org/journal/2007/08/quantifying-western-blots-without, using TIFF files.

Cell proliferation assays

HEK-293T/17 cell cytotoxicity induced by LASV Z, GPC, and NP expression was monitored with a TACS™ MTT Cell Proliferation Assay (R&D Systems, Minneapolis, Minn.), according to manufacturer's instructions. The transfection procedure was scaled down to a 96-well format, with each condition analyzed in triplicate. Data ere plotted as mean absorbance at 562 nm, with standard deviation, and background correction at 650 nm.

Protease protection assays

Pseudovirus-specific protein composition and VLP structure were characterized by trypsin protection assays. Ten μg of purified VLP was treated with 100 μg/mL trypsin in the presence or absence of 1% Triton® X-100, for 30 minutes, at room temperature. Reactions were stopped by the addition of soybean trypsin inhibitor to a final concentration of 3 mg/mL, addition of SDS-PAGE buffer and reducing agent (DTT), and heating to 70° C. for ten minutes. Proteins were resolved on 10% NuPage® gels and detected by western blot, as described above.

PNGase F, Endo H, and Neuraminidase assays

The glycosylation patterns of LASV VLP GP1 and GP2 generated from expression of LASV Z+GPC+NP were resolved by treatment with the deglycosidases PNGase F, Endo H, and Neuraminidase, as previously described (Branco et al., 2009), on sucrose cushion purified VLP. Reactions were performed on heat-denatured VLP to conform to manufacturer's recommendations for PNGase F and Endo H digestion conditions, and on non-denatured VLP. Control reactions were similarly processed except that enzymes were not added. Specificity of deglycosidases was assessed by monitoring the effects of all three enzymes on LASV NP and Z proteins packaged into VLP. Proteins were subsequently resolved by reducing SDS-PAGE, blotted, probed with anti-LASV GP1, GP2, or 6X-HIS mAbs, or goat anti-NP pAb, and developed as described above.

Lectin-based glycan differentiation assays

Glycosylation patterns of VLP associated proteins were characterized via binding of glycan-specific lectins using the DIG Glycan Differentiation Kit (Roche Applied Science, Mannheim, Germany) according to the manufacturer's instructions. LASV VLP proteins were resolved by reducing SDS-PAGE, blotted onto nitrocellulose, and subjected to lectin binding assays.

RNA extraction from purified VLP

RNA was extracted from VLP with Trizolm reagent/chloroform and isopropanol precipitation, essentially as outlined in the product insert (Invitrogen). RNA pellets were washed with 75% ethanol, air dried, resuspended in DEPC-treated water, and quantitated by A280. RNA was glyoxal-denatured and analyzed on 1.5% agarose gels containing ethidium bromide. Gels were photographed on a Kodak EDAS 120 system and images were saved as tiff files for densitometry analysis. Total RNA was extracted from corresponding transfected HEK293T/17 cells using the same procedure.

Genomic DNA fragmentation analysis

Genomic DNA was isolated from HEK-293T/17 cells using a Qiagen DNeasy kit, according to the manufacturer's instructions. Purified DNAs were quantitated by A260/A280. Two μg of each DNA sample was resolved per lane of a 1.8% TAE/agarose gel containing 1 μg/mL ethidium bromide. High resolution gel images were converted to tiff format for analysis.

Murine immunizations

Six to eight week-old female BALB/c mice were purchased from Charles River Laboratories. For immunizations, mice were randomly divided into groups of 10 and injected intraperitoneally with 10 μg of LASV VLP (Z+GPC or Z+GPC+NP) in 100 μL of sterile TNE. Ten mice were similarly injected with 100 μL TNE as vector control. One prime and two boosts were performed, three weeks apart, each with 10 μg of homologous LASV VLP. Mice were sacrificed by CO₂ asphyxiation three weeks after the last boost and whole blood was collected by cardiac puncture. The plasma fraction was isolated and frozen at -80° C. until analysis.

IgG and IgM ELISA on recombinant LASV proteins and VLP

Murine immunoglobulin-y endpoint titers to whole VLP, and IgG-y to GP1 and GP2 were determined in serially diluted sera samples. Nunc MaxiSorp® MaxiSorp ELISA plates were coated with 2 μg/mL total VLP protein in carbonate buffer. Recombinant mammalian cell-expressed LASV GP1 and GP2 proteins, produced by Vybion, Inc., Ithaca, N.Y., were coated on Nunc PolySorp® ELISA strips, pre-blocked, and lyophilized by Corgenix Medical Corp., Broomfield, Colo. Plates coated with VLP were blocked in 1× PBS, pH 7.4, 5% NFDM, 1% FBSΔ, 0.05% Tween®-20, 0.01% thymerosal. The same buffer was used for all sera and secondary antibody dilutions. Mouse IgG was detected with a horseradish peroxidase (HRP)-labeled goat F(ab')₂ anti-mouse IgG γ-specific reagent at 1:2500 dilution (KPL). Reactions were developed with TMB for 15 minutes at room temperature, stopped with 0.5 N H₂SO₄, and plates were read at 450 mn in a BioTek 808 ELISA reader. Viral antigen-specific IgG and IgM analysis in the sera of convalescent patients was similarly performed, with serum samples diluted 1:100 in NFDM blocking reagent, and detected with HRP-labeled goat F(ab')₂ anti-human IgG, γ- or μ-specific reagents, respectively. Monoclonal antibodies to GP2 and NP were used as positive controls on antigen-coated plates to verify presence of relevant epitopes on viral proteins. Total IgG fraction from naive mice was used as negative control antibody (ms IgG). Sera collected from North American volunteer blood donors that had never travelled to LHF endemic regions, and that were confirmed naive to LASV antigens by ELISA were used as negative controls. Serum from a patient that tested positive for NP-specific IgM and IgG antibodies in a recombinant NP ELISA was used as a positive control in these assays (G652-3).

Electron Microscopy

HEK-293T/17 cells were harvested at 72 hours post transfection with LASV gene constructs. Cells were pelleted by centrifugation at 200× g, washed once in cold (4° C.) PBS, and fixed with 2.5% glutaraldehyde in phosphate buffer. Fixed cell pellets were embedded in 1% agarose prepared in phosphate buffer and allowed to solidify at 4° C. Cell pellets in agarose were post fixed with 1% osmium tetroxide, dehydrated in a graded series of ethanol, and embedded in epoxy resin. Thin sections were cut on a Leica UC6 ultramicrotome, stained with uranyl acetate and lead citrate, followed by examination on a Hitachi H-7100 transmission electron microscope.

Statistical Analysis and in Silico Tools

Statistical analysis of data was performed with GraphPad InStat, V3.06 (GraphPad Software, Inc., San Diego, Calif.), using Analysis of Variance (ANOVA), paired or unpaired Student's t test, and Pearson's correlation. The PeptideCutter analysis tool from the Swiss Institute of Bioinformatics ExPASy Proteomics Server was employed in the in silico analysis of predicted trypsin cleavage sites on LASV GP1 and GP2.

### Example 6

Differential detection of LASV antigens in infected human patient sera

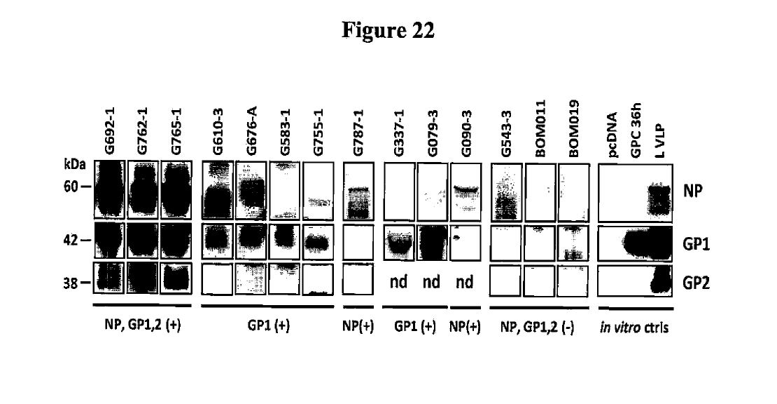

A total of 19 and 27 patient sera were analyzed in the first (Table 2) and second (Table 3) studies, respectively. A panel of serum samples from approximately 100 volunteer blood donors from the Northwestern district of Bombali, Sierra Leone, were analyzed for LASV-specific antigen, IgG and IgM antibodies, and designated BOM series. These samples were collected from individuals whose medical histories did not contain any indication of previous infection with Lassa virus. A significant percentage of BOM samples tested positive for IgM and IgG antibodies against LASV proteins in a recombinant ELISA, but all 100 were negative for virus antigen in a capture assay using an NP-specific detection platform (data not shown). A panel of archived human sera from patients that had been admitted to the KGH from January 2008-April 2010 as suspected Lassa fever cases were chosen for these studies. All archived samples containing sufficient quantities of serum (˜20-25 μL) with a diagnosis of “antigen positive” (Ag+) determined by a traditional Ag-capture assay (Trad Ag) developed by the United States Army Medical Research Institute of Infectious Diseases (USAMRIID), or a recombinant Ag-capture assay for LASV NP developed by the Hemorrhagic Fever Virus Diagnostics Consortium were used in this study. In addition, a subpanel of samples that were Ag-negative but were IgM- and/or IgG-positive, either by traditional LASV antibody platform (USAMRIID) [Trad IgG, Trad IgM] or recombinant protein-based (Hemorrhagic Fever Virus Diagnostics Consortium) [r IgG, r IgM] ELISA, were analyzed as controls. A profile of LASV antigens detected in serum samples analyzed in these studies is displayed in FIG. 22. Detection of all three antigens, NP, GP1, and GP2, could be detected at high levels in only three patient sera (FIG. 22, G692-1, G762-1, G765-1). All three patients succumbed to Lassa fever. In six independent patient sera only GP1 antigen was detected (FIG. 22, G610-3, G676-A, G583-1, G755-1, G337-1, G079-3), albeit to relatively low levels compared to the triple Ag-positive samples. Samples G337-1 and G079-3 were not analyzed for presence of the GP2 protein (FIG. 22). In two samples only NP antigen could be detected at low levels (FIG. 22, G787-1, G090-3), although GP2 analysis was not performed for the latter. In 34 patient and control sera none of the LASV proteins were detected (FIG. 22, G543-3, BOM011, BOM019). Identification of LASV proteins NP, GP1, and GP2 were aided by comparing to corresponding bands on VLP (FIG. 22, L VLP). The 42 kDa sGP1 component generated from expression of GPC in HEK-293T/17 cells is also shown (FIG. 22, GPC 36 h). Viral proteins could not be detected in supernatants of cells transfected with plasmid vector alone (FIG. 22, pcDNA).

TABLE 2^{a}

| | G079-3 | G090-2 | G090-3 | G106-1 | G153-1 | G165-1 | G193-1 | G327-2 | G337-1 | G408-2 | G418-11 | G443-12 |
|---|---|---|---|---|---|---|---|---|---|---|---|---|
| Trad Ag | + | + | 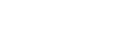 | − | − | − | + | + | + | − | − | 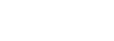 |
| Trad IgG | + | − | 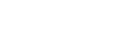 | + | +/− | 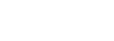 | − | 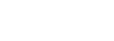 | − | − | 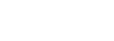 | 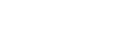 |
| Trad IgM | − | + | 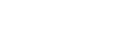 | + | + | + | − | − | − | + | − | 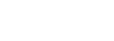 |
| rAg (NP) | + | − | − | 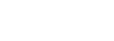 | 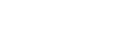 | 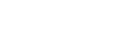 | + | 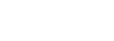 | + | 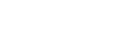 | − | 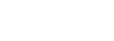 |
| rIgG | +/− | − | 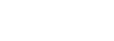 | + | + | 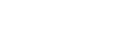 | − | 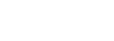 | +/− | + | 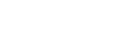 | 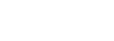 |
| rIgM | nd | nd | nd | nd | nd | 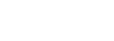 | nd | 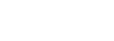 | nd | nd | 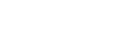 | 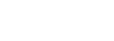 |
| GP1 WB | + | − | − | − | − | − | − | − | + | − | − | − |
| NP WB | − | − | + | − | − | − | − | − | − | − | − | − |

| | | | | | | | | | | | |
|---|---|---|---|---|---|---|---|---|---|---|---|
| outcome | D | d | d | D | D | D | d | na | d | d | d |

| | G540-4 | G551-3 | G579-1 | G598-2 | G590-1 | G610-1 | G617-1 | 293T ctrl | 293T/GP | L VLP |
|---|---|---|---|---|---|---|---|---|---|---|
| Trad Ag | + | + | − | − | + | + | + | 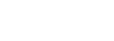 | 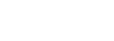 | 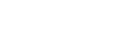 |
| Trad IgG | − | − | − | − | − | − | − | 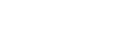 | 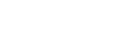 | 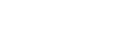 |
| Trad IgM | + | − | + | + | +/− | + | + | 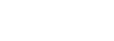 | 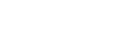 | 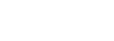 |
| rAg (NP) | 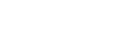 | 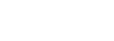 | − | − | 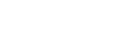 | − | − | 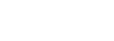 | 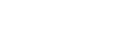 | 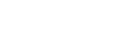 |
| rIgG | − | + | + | + | − | + | − | 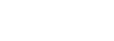 | 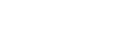 | 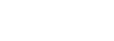 |
| rIgM | − | − | + | + | − | + | + | 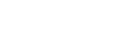 | 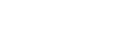 | 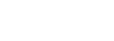 |
| GP1 WB | − | − | − | − | − | − | − | − | + | ++ |
| NP WB | − | − | − | − | − | − | − | − | − | ++ |
| outcome | d | d | D | na | D | d | d | na | na | na |

^{a}Trad Ag, IgG, IgM, rAg, rIgG, and rIgM were ELISA assays, whereas GP1 WB and NP WB were western blots. A very strong positive signal is indicated by ++, and a positive by +. Marginal positive detection is indicated by +/−, and negatives by −. Gray boxes indicate data not performed for a given assay. Patient outcomes, as recorded in available databases are: d, discharged; D, dead; na, not admitted.

TABLE 3^{a}

| | G543-3 | G548-1 | G583-1 | G598-2 | G610-3 | G645-2 | G652-3 | G676-A | G692-1 | G693-1 | G706-1 | G753-1 |
|---|---|---|---|---|---|---|---|---|---|---|---|---|
| Trad Ag | − | + | + | − | − | − | − | 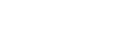 | + | − | + | + |
| Trad IgG | | | | | | | | | | | | |
| Trad | + | − | − | − | + | − | + | | − | − | − | − |
| IgM | | | | | | | | | | | | |
| rAg (NP) | | | + | − | − | − | +/− | 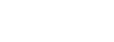 | + | − | + | + |
| rIgG | 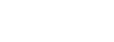 | 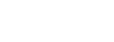 | 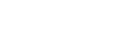 | 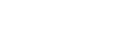 | 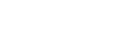 | 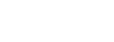 | + | 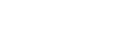 | + | +/− | +/− | +/− |
| rIgM | 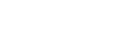 | 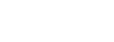 | +/− | + | 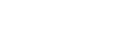 | + | + | 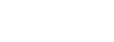 | + | − | +/− | +/− |
| GP1 WB | − | − | + | − | + | − | − | + | ++ | − | − | − |
| GP2 WB | − | − | − | − | − | − | − | − | + | − | − | − |
| NP WB | − | − | − | − | − | − | − | − | ++ | − | − | − |
| outcome | d | D | d | na | D | d | d | c | D | D | d | d |
| | G755-1 | G756-1 | G762-1 | G765-1 | G771-1 | G784-1 | G787-1 | G793-1 | G795-1 | G802-1 | G803-1 | G803-2 |
| Trad Ag | − | + | + | + | + | + | − | − | + | + | − | − |
| Trad IgG | | | | | | | | | | | | |
| Trad | − | − | − | − | − | − | − | − | − | − | + | − |
| IgM | | | | | | | | | | | | |
| rAg | − | +/− | + | + | + | + | − | − | − | − | +/− | +/− |
| (NP) | | | | | | | | | | | | |
| rIgG | + | + | ++ | + | +/− | +/− | +/− | + | + | + | + | + |
| rIgM | + | +/− | − | + | − | + | + | + | + | + | ++ | +/− |
| GP1 WB | + | − | ++ | ++ | − | − | − | − | − | − | − | − |
| GP2 WB | − | − | ++ | ++ | − | − | − | − | − | − | − | − |
| NP WB | − | − | ++ | ++ | − | − | +/− | − | − | − | − | − |
| outcome | na | d | D | ? | D | D | ? | d | ? | d | d | d |

| | G804-1 | G806-1 | G808-1 | BOM011 | BOM019 | 293T ctrl | 293T/GP | L VLP |
|---|---|---|---|---|---|---|---|---|
| Trad Ag | + | + | + | 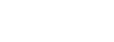 | 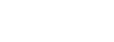 | 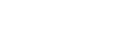 | 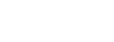 | 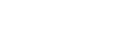 |
| Trad IgG | | | | 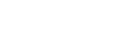 | 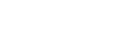 | 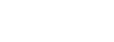 | 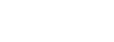 | 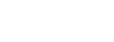 |
| Trad IgM | − | − | − | 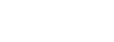 | 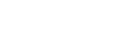 | 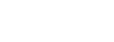 | 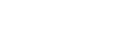 | 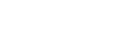 |
| rAg (NP) | | − | + | − | − | 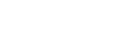 | 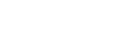 | 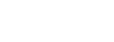 |
| rIgG | ++ | + | + | + | 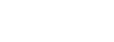 | 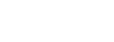 | 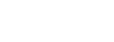 | 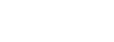 |
| rIgM | ++ | ++ | − | − | 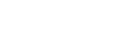 | 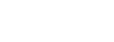 | 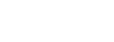 | 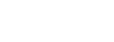 |
| GP1 WB | − | − | − | − | − | − | + | ++ |
| GP2 WB | − | − | − | − | − | − | − | ++ |
| NP WB | − | − | − | − | − | − | − | ++ |
| outcome | d | D | d | 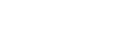 | 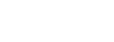 | 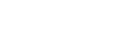 | 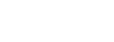 | 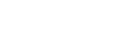 |

^{a}Trad Ag, IgG, IgM, rAg, rIgG, and rIgM were ELISA assays, whereas GP1 WB, GP2 WB, and NP WB were western blots. A very strong positive signal is indicated by ++, and a positive by +. Marginal positive detection is indicated by +/−, and negatives by −. Gray boxes indicate data not performed for a given assay. Patient outcomes, as recorded in available databases are: d, discharged; D, dead; na, not admitted; c, household contact of patient G676; ?, patient samples from Liberia, without unknown outcome.

Discussion

In previous studies, we described and characterized the phenomenon of LASV GP1 ectodomain shedding in vitro (Branco and Garry, 2009, Virol. J. 6:147). In order to investigate whether this phenomenon was specific in vitro we analyzed serum samples from patients admitted to the KGH in Sierra Leone from 2008-2010 for the differential detection of LASV proteins. Our approach involved the detection of GP1, GP2, and NP in the same sample, with sensitive monoclonal and polyclonal antibodies. The detection of NP alone could be indicative of release of the protein from virally infected dead cells, and could conceivably remain in the bloodstream after viral clearance. This phenomenon has been observed in vitro. Thus, acute viremia was characterized as the concomitant detection of both GP1 and GP2, and the nucleoprotein. The detection of GP2 implies its presence in the context of an enveloped virion, due to its known membrane-spanning properties via the transmembrane domain (amino acids 427-451 in LASV Josiah), whereas GP1 would be present as a component of the non-covalently linked tripartite GPC complex. The nucleoprotein component of the virion should also be detected in all samples containing GP1 and GP2, thus confirming the presence of intact, enveloped, circulating Lassa virions. The sole detection of GPI in any given sample was interpreted as an absence of whole virions and presence of the soluble form of the protein, as previously observed in vitro. The time of collection of any given blood sample represents a snapshot in the stage of a potential LASV infection. Within the context of an early acute viral infection it is unlikely that a patient would present with symptoms immediately following exposure to the virus. Following viral infection of host cells and early replication events, the detection of sGP1 without accompanying progeny virions might be possible and was therefore tested. This event may represent a very narrow window in the virus life cycle in vivo. Thus, the ratio of LASV-positive samples in which sGP1 alone was detected was small (6/46) in the present studies. Following this very early step in viral biogenesis, progeny virions will emerge from the surface of infected cells and will disseminate throughout body tissues and fluids. At this stage it will no longer be possible to differentiate shedded sGP1 from virion-associated GP1. Detection of GP1, GP2, and NP in IgM and/or IgG-positive samples also falls outside the window of detection of the sGP1 component, as presence of these immunoglobulins represent a more advanced stage in the course of the disease. In the cases where viral antigens, IgM and IgG were detected (G692-1, G762-1, G765-1) the possibility exists that a re-infection scenario with clinical symptoms and development of febrile disease occurred. Two out of these three patients succumbed to Lassa fever, whereas the outcome of one patient (G765-1) is not known. Each sample analyzed in these studies for LASV antigens was exhaustively subjected to western blot analysis, with different viral protein-specific antibody reagents, at different serum dilutions, with extended exposures to sensitive X-ray films, and using an extensive panel of positive and negative controls, to ensure that data were not the result of artifacts or background noise. Therefore, each sample was analyzed for presence of each antigen at least three times, either in a primary detection or in probing and reprobing formats. Although probing and reprobing allowed for the detection of antigens using a single blot, and thus preserving precious sample volumes, the membrane stripping process removes protein from the matrix, thus reducing the sensitivity of subsequent assays. The small volumes of available serum for analysis made further characterization of each sample unfeasible.

In these studies three sets of relevant data were used to interpret the antigenic status of the serum samples: 1. Traditional antigen (Trad Ag) ELISA; 2. Recombinant antigen capture, NP-specific; 3. Western blot analysis of GP1, GP2, and NP. Direct correlations cannot be made between the three assay platforms based on several factors. The USAMRIID antigen capture assay employs two GP1-, two GP2-, and one NP-specific mAbs, thus individual identification of each antigen cannot be established. Furthermore, this assay relies on a two-step signal amplification and detection, each with polyclonal antibody reagents, a rabbit secondary and a goat tertiary. Thus, the sensitivity of this assay may be superior to the NP mAb-based antigen capture format, which employs a single detection step with a goat polyclonal raised against recombinantly expressed NP protein. Both antigen capture formats were performed with 1:10 dilutions of serum in assay buffer, in a total of 100 μL. Western blots were performed with precipitated protein from 20 μL of whole serum, or 1:4 dilutions of equivalently processed samples, or 5 μL total serum. Each analytical format detects antigens in different conformations, thus direct data comparison is not feasible. Despite specificity and sensitivity issues at play, in 21/30 samples for which data is available from all three assay formats, the results from the Trad Ag and rAg platforms agreed. In six samples the Trad Ag assay detected LASV antigen, whereas the rAg platform did not. The higher sensitivity of the former may be due to the combination of 3 antigen-specific mAbs that detected LASV glycoproteins in addition to NP in the Trag Ag assay. Another possibility to consider is the quality of stored samples in less than ideal conditions over extended periods of time. The lack of continuous electrical power at the KGH over the years has resulted in fluctuations in temperature in refrigerators and freezers, thus quite possibly interfering with sample quality and stability of viral antigens. The time elapsed between collection and analysis of samples used in these studies varied between 3 months and 2 years. However, none of these six samples tested positive for sGP1 by western blot (G090-2, G610-1, G617-1, G795-1, G802-1, G806-1). In three additional samples, the rAg assay detected NP in serum samples marginally (G652-3, G803-1, G803-2). Neither of the three samples tested positive for sGP1 or NP by western blot. Three samples that tested positive in the Trag Ag and rAg ELISA assays, along with detection of sGP1, but not NP, were not considered as examples of glycoprotein shedding (G079-3, G337-1, G583-1). In only one of these three samples detection of GP2 was performed in addition to GP1 and NP (G583-1). The clear detection of GP1 in G583-1 would imply similar levels of GP2 in the context of a circulating virion, in addition to NP. Despite absence of GP2 the presence of NP obscured the clear distinction of shed GP1 prior to viral biogenesis. Thus, we have considered only samples G610-3 and G775-1 as the only examples of clear LASV GP1 shedding in these studies. In both samples LASV antigen was not detected by Trag Ag and rAg ELISA, and GP2 and NP western blot platforms, whereas GPI was clearly present (FIG. 22, G610-3, G755-1). For sample G676-A, which was collected from a household contact of patient G676 who succumbed to Lassa fever, Trag Ag and rAg ELISA data were not available. This sample was also not considered a clear example of GP1 shedding.

Based on the proposed narrow window of sGP1 detection in vivo, which is likely to occur in the first few days after primary infection with LASV, it is not surprising that only 2/46 samples analyzed in these studies would show a clear distinction of early LASV infection events. The known incubation period for infection with LASV is approximately 6-7 days, with longer and shorter times reported, but admission to local hospitals is usually delayed following the onset of febrile disease. Thus, in many cases patients admitted to the KGH present with acute viremia or undetectable antigen but rising IgM titers, indicative of advanced LASV infection.

Strecker et al. (2003, J. Virol. 77:10700) reported the stoichiometric ratio of NP:GP1:GP2 in Lassa virions as 160:60:60. In the context of a LASV virion, detection of one protein should result in the detection of the other two. Thus, detection of GP1 without concomitant detection of either GP2 or NP can be interpreted as a secreted isoform of GP1 prior to the emergence of enveloped viral particles from infected cells, that likely corresponds to the sGP1 component identified in vitro (FIG. 22).

We have identified through this work the presence of a soluble form of LASV GP1 in the serum of infected patients. Although the exact stage of viral infection could not be determined in these studies, the lack of detection of the virion-associated nucleoprotein and GP2 proteins, with clear identification of GP1 in the serum of acutely infected individuals, points to an early event in viral replication when only sGP1 can be detected. Although the role of a secreted GP1 component in arenaviral infections has not been established, it is possible that it performs immunomodulatory functions similar to those proposed for EBOV. Furthermore, therapeutic intervention at earlier times after onset of infection may mitigate the usually poor outcome associated with Lassa hemorrhagic fever. Definition of the role(s) of LASV sGP1 in vivo could lead to new correlates of the disease, opportunities toward development of diagnostics targeting very early events in acute infection and viral biogenesis, and the ability to counter potential viral immunomodulatory pathways that confer poor outcomes.

Methods

Precipitation of total protein from human serum samples

Serum samples collected from patients admitted to the KGH Lassa fever ward (G-series), household contacts of hospitalized subjects (G-series-A), and individuals not known to have had Lassa fever (BOM) were aliquoted and stored at −20° C. in cryovials at the KGH Lassa fever laboratory. Twenty μL of each serum sample was diluted 5-fold with sterile D-PBS, pH 7.4 and combined with 20% polyethylene glycol-6000 (PEG-6000) and 2 M NaCl stock solutions to final concentrations of 5% and 0.2M, respectively. Samples were incubated at 4° C. overnight, followed by centrifugation at 21,000× g, for 75 minutes at 4° C. Supernatants were carefully aspirated and discarded. Pellets were resuspended in SDS-PAGE buffer with 50% glycerol, heated without reducing agent, and stored frozen until shipment. Samples were shipped to the U.S. in IATA-approved containers and were irradiated with 2500 KRad upon arrival, using a Cs source. Recombinant LASV VLP expressing Z+NP+GPC were used as controls for identification of viral proteins in SDS-PAGE, along with soluble GP1 (sGP1) from HEK-293T/17 cells transfected with a wild type GPC gene.

Western Blot Analysis

Four-fold dilutions of protein sample from a 20-μL serum aliquot were prepared in SDS-PAGE sample buffer, reduced with DTT, heated to 75° C. for 10 minutes, and resolved on 10% NuPAGE® Novex Bis-Tris gels, according to the manufacturer's specifications (Novex, San Diego, Calif.). Proteins were transferred to 0.45-μm nitrocellulose membranes, blocked, and probed in 1× PBS, pH 7.4, 5% non-fat dry milk, 1% heat inactivated fetal bovine serum, 0.05% Tween®-20, and 0.1% thymerosal. Detection of LASV GP1, GP2, and NP in precipitated protein from human serum samples was performed by Western blot analysis using anti-LASV mAbs L52-74-7A (GP1), L52-216-7 and L52-272-7 (GP2), and goat pAb to E. coli-generated nucleoprotein, respectively. Secondary antibodies were horseradish peroxidase (HRP)-conjugated goat anti-mouse IgG (H+L) or rabbit anti goat IgG (H+L). Membranes were then incubated in LumiGlo® chemiluminescent substrate (KPL) and exposed to HyBlot® CL Film (Denville Scientific, Inc). Blots used in reprobing experiments were briefly rinsed in PBS-T (1X PBS, pH 7.4, 0.1% Tween® 20) after exposure to X-ray film, followed by incubation in stripping buffer (62.5 mM Tris, pH 6.8, 2% SDS, 100 mM n-ME) for one hour at 65° C. Blots were then washed extensively in PBS-T, re-blocked, and reprobed as outlined above. Blots were reprobed a maximum of three times.

All patents and publications identified in this application are hereby incorporated by reference in their entirety.

1. A nucleic acid expression construct for producing an arenavirus-like particle, wherein the construct comprises:
a promoter, and
sequences encoding (i) a first protein comprising an arenavirus matrix (Z) protein, and (ii) a second protein comprising a different arenavirus protein;
wherein said promoter is heterologous with respect to at least one of said sequences.

2. The construct of claim 1, wherein the second protein comprises an arenavirus glycoprotein precursor (GPC) protein, an arenavirus nucleoprotein (NP), an arenavirus glycoprotein-1 (GP1) protein, or an arenavirus glycoprotein-2 (GP2) protein.

3. The construct of claim 2, wherein the construct further comprises a sequence encoding a third protein, and wherein the second and third proteins comprise, respectively, arenavirus GPC and NP proteins.

4. The construct of claim 2, wherein the construct further comprises a sequence encoding a third protein, and wherein the second and third proteins comprise, respectively, arenavirus GP1 and GP2 proteins.

5. The construct of claim 1, wherein at least one arenavirus protein encoded by the construct is derived from Lassa virus.

6. The construct of claim 1, wherein each arenavirus protein-encoding sequence of the construct is comprised within its own expression cassette, wherein each expression cassette comprises a promoter and a transcription termination sequence.

7. The construct of claim 1, wherein said construct is a eukaryotic expression construct.

8. A method of preparing an arenavirus-like particle comprising:
providing a nucleic acid expression construct according to claim 1, and introducing said construct into a eukaryotic cell to express said first and second proteins.

9. The method of claim 8, wherein said cell is a mammalian cell.

10. An arenavirus-like particle comprising (i) a first protein comprising an arenavirus matrix (Z) protein, and (ii) a second protein comprising an arenavirus nucleoprotein (NP).

11. The arenavirus-like particle of claim 10, wherein the particle further comprises a third protein comprising an arenavirus glycoprotein precursor (GPC) protein, an arenavirus glycoprotein-1 (GP1) protein, or an arenavirus glycoprotein-2 (GP2) protein.

12. The arenavirus-like particle of claim 11, wherein the third protein comprises an arenavirus GPC protein.

13. The arenavirus-like particle of claim 11, wherein the third protein comprises an arenavirus GP1 or GP2 protein.

14. The arenavirus-like particle of claim 10, wherein at least one arenavirus protein comprised within said particle is derived from Lassa virus.

15. A vaccine comprising an arenavirus-like particle according to claim 10.

16. A method of diagnosing an LASV infection before the onset of febrile disease, wherein said method comprises detecting LASV GP1 protein in the blood of an individual without likewise detecting other LASV proteins.
